(19) United States
(12) Patent Application Publication
(10) Pub. No.: US 2025/0339126 A1
(43) Pub. Date: Nov. 6, 2025

(54) MONITORING KIDNEY PERFUSION USING ULTRASOUND

(71) Applicant: Becton, Dickinson and Company, Franklin Lakes, NJ (US)

(72) Inventors: Blake W. Axelrod, South Pasadena, CA (US); Mark A. Konno, Laguna Beach, CA (US); Julio Agustin Isla Garcia, Irvine, CA (US); Andres S. Aguirre, Mission Viejo, CA (US); Robert Bruce Alexander Adamson, Halifax (CA); Chris Samson, Halifax (CA)

(21) Appl. No.: 19/265,980

(22) Filed: Jul. 10, 2025

Related U.S. Application Data
(63) Continuation of application No. PCT/US2024/011065, filed on Jan. 10, 2024.
(60) Provisional application No. 63/479,254, filed on Jan. 10, 2023.

(51) Int. Cl. A61B 8/00; A61B 8/06

(52) U.S. Cl. CPC A61B 8/4236; A61B 8/06; A61B 8/4488; A61B 8/4494; A61B 8/461; A61B 8/488; A61B 8/5223

(57) ABSTRACT

A blood flow monitor includes an ultrasound transducer probe with a two-dimensional array of transducer elements. An adhesive patch is connected to the ultrasound transducer probe and is configured to attach the ultrasound transducer probe to a patient. The blood flow monitor also includes a system memory that stores beamformer software code. A processor is in communication with the ultrasound transducer probe and the system memory. The processor is configured to execute the beamformer software code to steer a beam to scan the patient with the array of transducer elements to find a Doppler flow signal of a targeted blood flow of the patient.

### CROSS-REFERENCE TO RELATED APPLICATION(S)

This application is a continuation of International Application No. PCT/US2024/011065, filed Jan. 10, 2024, entitled “MONITORING KIDNEY PERFUSION USING ULTRASOUND,” which claims the benefit of U.S. Provisional Application No. 63/479,254, filed Jan. 10, 2023, and entitled “MONITORING KIDNEY PERFUSION USING ULTRASOUND,” the disclosures of which are hereby incorporated by reference in their entireties.

### BACKGROUND

Acute kidney injury (AKI) occurs when a kidney experiences a sudden decrease in function. AKI can be a complication from major abdominal surgery and may increase a risk of chronic kidney disease in a patient if AKI is not detected and treated at an early stage. Decreased perfusion to the kidney(s) during surgery is one cause of AKI. Detecting AKI in a patient is traditionally done by viewing two biomarkers in the patient. The first biomarker is analyzing urine output of the patient and the second biomarker is measuring serum creatinine from a blood sample of the patient. These biomarkers generally do not show up in the patient until about eight hours to forty-eight hours after the injury has occurred to the kidney(s). Due to the late onset of these biomarkers, physicians can only use these biomarkers to detect whether AKI has occurred a relatively long time after the kidney has been damaged and cannot use these biomarkers to monitor health of the kidneys in real time during a surgery. The ability to monitor the health of the kidneys during surgery would not only allow physicians the ability of early detection of AKI, but possibly the ability to prevent AKI in the patient.

### SUMMARY

A renal blood flow monitor includes an ultrasound transducer probe with a two-dimensional array of transducer elements. An adhesive patch is connected to the ultrasound transducer probe and is configured to attach the ultrasound transducer probe to a patient and maintain contact between the patient and the ultrasound transducer probe without an operator. The renal blood flow monitor also includes a beamformer to drive the two-dimensional array of transducer elements. The beamformer is configured to cause the two-dimensional array of transducer elements to emit multiple ultrasound beams from the two-dimensional array of transducer elements to track a Doppler flow signal of a renal blood flow of the patient relative to the array of transducer elements.

A method is disclosed for monitoring renal blood flow of a patient. The method includes positioning an ultrasound transducer probe on an abdomen of the patient. The ultrasound transducer probe comprises a two-dimensional array of transducer elements. The abdomen of the patient is scanned with the two-dimensional array of transducer elements and a beamformer drives the array of transducer elements to find and sense a Doppler flow signal of the renal blood flow of the patient. The ultrasound transducer probe is attached to the abdomen of the patient by an adhesive patch connected to the ultrasound transducer probe. The ultrasound transducer probe is at a position on the abdomen of the patient where the Doppler flow signal of the renal blood flow of the patient was found. The beamformer and the array of transducer elements track-scan the Doppler flow signal of the renal blood flow of the patient to continuously sense the Doppler flow signal of the renal blood flow of the patient during a surgery, medical procedure, or medical observation without an ultrasound operator.

An organ blood flow monitor includes an ultrasound transducer probe with a two-dimensional array of transducer elements. An adhesive patch is connected to the ultrasound transducer probe and is configured to attach the ultrasound transducer probe to a patient. The organ flow monitor also includes a beamformer to drive the two-dimensional array of transducer elements. The beamformer is configured to cause the two-dimensional array of transducer elements to emit multiple ultrasound beams from the two-dimensional array of transducer elements to track a targeted organ blood flow signal relative to the array of transducer elements.

A method is disclosed for monitoring organ blood flow of a targeted organ of a patient during a surgery, medical procedure, or medical observation. The method includes positioning an ultrasound transducer probe on an abdomen of the patient. The ultrasound transducer probe comprises a two-dimensional array of transducer elements. A beamformer drives the array of transducer elements to scan a targeted organ location of the patient to find a Doppler flow signal of the organ blood flow of the patient. An adhesive patch connected to the ultrasound transducer probe attaches the ultrasound transducer probe to the patient at the targeted organ location where the Doppler flow signal of the organ blood flow of the patient was found. The beamformer track-scans the Doppler flow signal of the organ blood flow of the patient to continuously sense the Doppler flow signal of the organ blood flow of the patient without repositioning the ultrasound transducer probe during the surgery, medical procedure, or medical observation.

A renal blood flow monitor includes an ultrasound transducer probe and an adhesive patch connected to the ultrasound transducer probe for attaching the ultrasound transducer probe to a patient. The renal blood flow monitor also includes an organ recognition algorithm configured to distinguish a renal blood flow signal from a non-renal blood flow signal based upon waveform characteristics of the renal blood flow signal.

A renal blood flow monitor includes an ultrasound transducer probe with an array of transducer elements. An adhesive patch is connected to the ultrasound transducer probe and is configured to connect the ultrasound transducer probe to a patient. The renal blood flow monitor also includes a system memory that stores beamformer software code. A processor is in communication with the system memory and a control module of the ultrasound transducer probe. The processor is configured to execute the beamformer software code to beam scan the patient with the array of transducer elements to find a Doppler flow signal of a renal blood flow of the patient.

A blood flow monitor includes an ultrasound transducer probe with a two-dimensional array of transducer elements. An adhesive patch is connected to the ultrasound transducer probe and is configured to attach the ultrasound transducer probe to a patient. The blood flow monitor also includes a system memory that stores beamformer software code. A processor is in communication with the system memory and a control module of the ultrasound transducer probe. The processor is configured to execute the beamformer software code to steer a beam to scan the patient with the array of transducer elements to find a Doppler flow signal of a targeted blood flow of the patient.

The present summary is provided only by way of example, and not limitation. Other aspects of the present disclosure will be appreciated in view of the entirety of the present disclosure, including the entire text, claims and accompanying figures.

### BRIEF DESCRIPTION OF THE DRAWINGS

FIG. 1 is a schematic diagram illustrating an example blood flow monitor with an ultrasound transducer probe attached to an abdomen of a patient by an adhesive patch.

FIG. 2 is another schematic diagram illustrating the blood flow monitor of FIG. 1.

FIG. 3 is a schematic diagram of an ultrasound transducer probe attached to an abdomen of a patient by an adhesive patch to monitor a kidney of the patient.

FIG. 4A is another schematic diagram of an ultrasound transducer probe attached to an abdomen of a patient by an adhesive patch to monitor a kidney of the patient.

FIG. 4B is another schematic diagram of an ultrasound transducer probe attached to an abdomen of a patient by an adhesive patch to monitor a kidney of the patient.

FIG. 5 is a schematic diagram of an ultrasound transducer probe with an array of transducer elements.

FIG. 6 is a schematic diagram illustrating another example blood flow monitor with two ultrasound transducer probes attached to an abdomen of a patient to monitor both kidneys of the patient.

FIG. 7 is a schematic diagram illustrating two blood flow monitors and two ultrasound transducer probes attached to an abdomen of a patient to monitor blood flow in a kidney and blood flow in a liver of the patient.

FIG. 8 is a chart from an experiment showing a first plot of a renal blood flow measured by the blood flow monitor of FIG. 1 compared to both a second plot of the renal blood flow measured by an invasive transonic flow probe and a plot of mean arterial blood pressure (MAP).

FIG. 9 is a schematic diagram illustrating another example blood flow monitor with an ultrasound transducer probe attached to an abdomen of a patient by an adhesive patch.

FIG. 10 is another schematic diagram illustrating the blood flow monitor of FIG. 9.

FIG. 11 is a block diagram of a method for continuously monitoring a blood flow of an organ of a patient.

FIG. 12 is a block diagram of another method for continuously monitoring a blood flow of an organ of a patient.

FIG. 13 is a schematic diagram illustrating another example blood flow monitor.

### DETAILED DESCRIPTION

The present disclosure is directed to a system and a method to monitor in real time a blood flow of an abdominal organ, such as a kidney, of a patient during a surgery, medical procedure, or medical observation of the patient. The system includes a blood flow monitor with an ultrasound transducer probe. The system also includes an adhesive patch that can attach the ultrasound transducer probe to a patient and keep the ultrasound transducer probe attached to the patient through a surgery, medical procedure, or medical observation of the patient without assistance from an ultrasound operator. The blood flow monitor also includes a beamformer and ultrasound front-end (UFE) circuitry in communication with the ultrasound transducer probe to drive an array of transducer elements of the ultrasound transducer probe.

In this disclosure, a Doppler flow signal is defined as comprising an ultrasound pulse-echo signal received from tissue, filtered to only contain those spectral components with a large enough Doppler shift to be reliably identified as having been generated by flowing blood cells. An instantaneous spectrum is defined as a power spectrum of a windowed portion of the Doppler flow signal with a window centered at a particular moment in time. In this disclosure, a Doppler spectrogram is defined as a time-frequency representation of the Doppler flow signal in which instantaneous spectrum is calculated for many timepoints to characterize how the instantaneous spectrum changes over time. The Doppler spectrogram is often visualized as a heat-map plot with frequency along one axis and time along a second axis. Relative intensity of the Doppler spectrogram can be interpreted as an indication of a fraction of scatterers with a particular velocity (i.e. a particular Doppler shift) at a particular moment in time. Negative frequency components of the Doppler spectrogram arise from scatterers that move away from the ultrasound transducer probe while the positive frequency components arise from scatterers moving towards the ultrasound transducer probe. Integrated power spectrum is defined as comprising the integral of the Doppler spectrogram along a frequency dimension. The integral of the Doppler spectrogram may be taken over all frequencies, over only the positive frequencies, over only the negative frequencies or over some other subset of frequencies. In cases where a signal from a particular vessel is sought, the integrated power spectrum will be calculated over a range of frequencies appropriate to isolate the Doppler flow signal from that vessel from interfering signals of nearby vessels. In particular, since blood flow in the renal artery is directed towards the ultrasound transducer probe and blood flow in the renal vein is directed away from the ultrasound transducer probe, the integrated power spectrum calculated in relation to the renal artery can comprise an integral over only positive frequencies while the integrated power spectrum calculated in relation to the renal vein can be calculated only over negative frequencies.

Depending on the application, the system may be configured to measure flow in many multiple different arteries or veins in various organs using the same techniques described in this disclosure for scanning, tracking and measuring Doppler signals. When methods are not specific to a particular vessel, the vessel that is being tracked will be referred to as the target vessel.

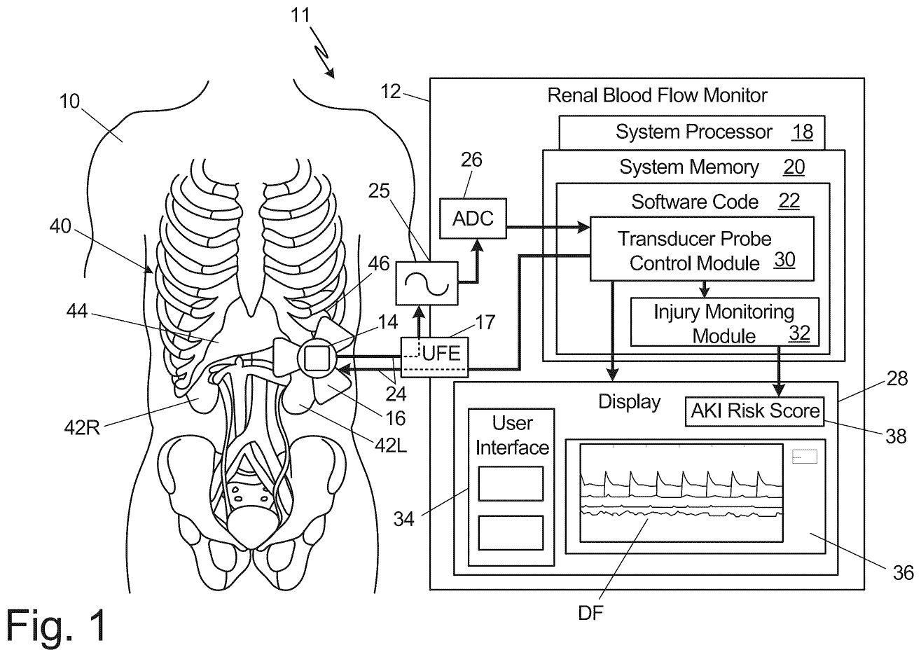

The beamformer is configured to continuously track a Doppler flow signal of an organ blood flow, such as renal blood flow, of the patient by emitting a set of sequential beams from the array of transducer elements to track the Doppler flow signal of the organ blood flow relative to the array of transducer elements focused on different locations. By tracking the Doppler flow signal of the organ blood flow, the beamformer allows the ultrasound transducer probe to continuously sense the Doppler flow signal of the organ blood flow throughout the surgery, medical procedure, or medical observation without moving or readjusting the position of the ultrasound transducer probe on the patient. Even if the organ shifts position in the abdomen of the patient, the beamformer enables the ultrasound transducer probe to continue sensing the organ blood flow without moving or readjusting the position of the ultrasound transducer probe on the patient. The ultrasound transducer probe sends a real time continuous reading of the organ blood flow to the blood flow monitor for health monitoring and perfusion of the organ through the duration of the surgery, medical procedure, or medical observation. The blood flow monitoring system is described in detail below with reference to FIGS. 1-13.

FIG. 1 is a schematic diagram of patient 10 and monitoring system 11 that continuously monitors an organ blood flow of patient 10 during a surgery, medical procedure, or medical observation. As shown in the example of FIG. 1, monitoring system 11 can include renal blood flow monitor 12, ultrasound transducer probe 14, adhesive patch 16, ultrasound front-end circuitry 17, system processor 18, system memory 20 with software code 22, probe cables 24, analog-to-digital (ADC) converter 26, and display 28. Software code 22 can include transducer probe control module 30 and injury monitoring module 32. Display 28 can include user interface 34, plot 36, and injury score indicator 38. FIG. 1 also shows abdomen 40 of patient 10 along with kidneys 42L and 42R, liver 44, and spleen 46. In the example of FIG. 1, monitoring system 11 is monitoring a renal blood flow of kidney 42L of patient 10. In other examples, monitoring system 11 can be used to monitor hepatic blood flow of liver 44, to monitor celiac blood flow of spleen 46, the pancreas (not shown), and the stomach (not shown) of patient 10, to monitor mesenteric blood flow of the intestines, and/or to monitor portal blood flow from the stomach of patient 10. Thus, renal blood flow monitor 12 can be adapted as an organ blood flow monitor 12 for any abdominal organ of patient 10.

Renal blood flow monitor 12, can be, e.g., an integrated hardware unit that includes system processor 18, system memory 20, display 28, ultrasound front-end circuitry 17, and ADC 26. In other examples, any one or more components and/or described functionality of organ blood flow monitor can be distributed among multiple hardware units. For instance, in some examples, display 28 can be a separate display device that is remote from and operatively coupled with renal blood flow monitor 12. In general, though illustrated and described in the example of FIG. 1 as an integrated hardware unit, it should be understood that renal blood flow monitor 12 can include any combination of devices and components that are electrically, communicatively, or otherwise operatively connected to perform functionality attributed herein to renal blood flow monitor 12.

Ultrasound transducer probe 14 can be attached or secured to patient 10 by adhesive patch 16. In the example of FIG. 1, ultrasound transducer probe 14 is positioned on abdomen 40 of patient 10 over at least a portion of kidney 42L. Adhesive patch 16 can include a sheet of structural material, such as fabric or flexible plastic, with a layer of bonding adhesive deposited on a face of the sheet. Adhesive patch 16 can be bonded to or mechanically connected to ultrasound transducer probe 14, or to a frame (not shown) connected to a base of ultrasound transducer probe 14, and can extend outward from ultrasound transducer probe 14 along a surface of abdomen 40 of patient 10. In other examples, adhesive patch 16 can be placed over ultrasound transducer probe 14 to attach ultrasound transducer probe 14 to abdomen 40 of patient 10. Adhesive patch 16 keeps ultrasound transducer probe 14 attached to patient 10 and secured in place throughout a duration of the surgery, medical procedure, or medical observation of patient 10. Since adhesive patch 16 keeps ultrasound transducer probe 14 immobile and in contact with patient 10, an ultrasound operator or technician is not needed during the surgery, medical procedure, or medical observation to keep ultrasound transducer probe 14 in position. A coupling layer (not shown) with a couplant material can be positioned between a skin of patient 10 and ultrasound transducer probe 14. The coupling layer enables ultrasonic energy transmission between the skin of patient 10 and ultrasound transducer probe 14.

In the example of FIG. 1, the ultrasound transducer probe 14 detects and senses a Doppler flow signal DF of the renal blood flow of kidney 42L. Ultrasound transducer probe 14 can be operatively connected to renal blood flow monitor 12 by cables 24. Via cables 24, ultrasound transducer probe 14 can receive electrical signals from the ultrasound front-end circuitry 17 of the renal blood flow monitor 12 and can relay the received ultrasound signals from patient 10 to renal blood flow monitor 12 for extraction of the Doppler flow signal DF of the renal blood flow of kidney 42L. In other examples, ultrasound front-end circuitry 17 is combined with ultrasound transducer probe 14, can be battery powered and can include a receiver to wirelessly receive commands from renal blood flow monitor 12. The combined ultrasound front-end circuitry 17 and ultrasound transducer probe 14 can also include a transmitter to wirelessly communicate the Doppler flow signal DF of the renal blood flow of kidney 42L to renal blood flow monitor 12 for analysis. In some examples, the combined ultrasound transducer probe 14 and ultrasound front-end circuitry 17 provide the Doppler flow signal DF to renal blood flow monitor 12 as analog signal 25, which is converted by ADC 26 to digital hemodynamic data representative of the renal blood flow of kidney 42L. In other examples, the combined ultrasound transducer probe 14 and ultrasound front-end circuitry 17 can provide the sensed Doppler flow signal DF to renal blood flow monitor 12 in digital form, in which case renal blood flow monitor 12 may not include or utilize ADC 26. In yet other examples, ultrasound transducer probe 14 can provide the Doppler flow signal DF of the renal blood flow of kidney 42L to blood flow monitor 12 as analog signal 25, which is analyzed in its analog form by blood flow monitor 12.

System memory 20 can be configured to store information within renal blood flow monitor 12 during operation. System memory 20, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 20 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

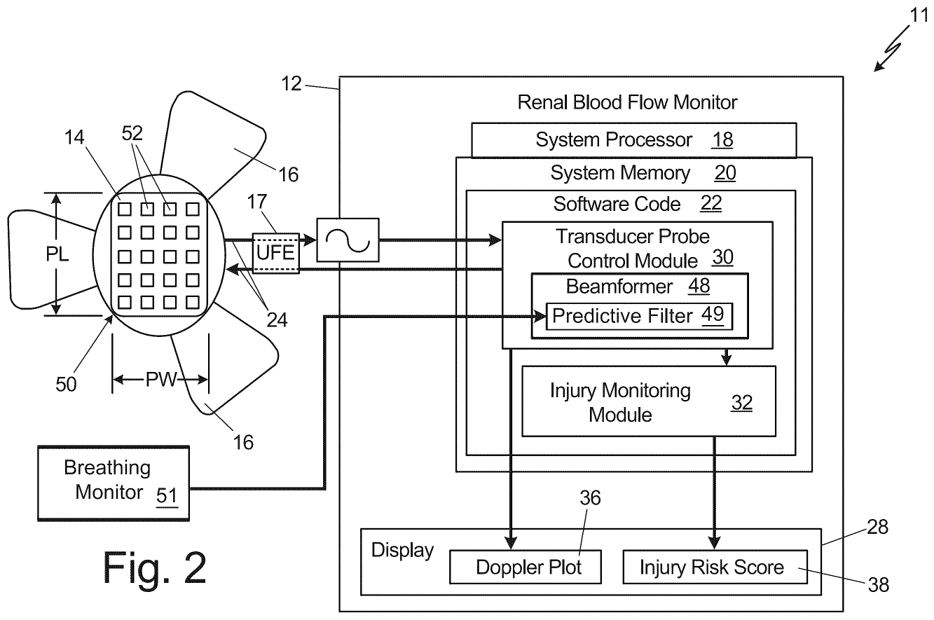

As shown in FIG. 1, system memory 20 of renal blood flow monitor 12 can store software code 22 which forms a monitoring model of renal blood flow monitor 12. Software code 22 can include transducer probe control module 30 for controlling and commanding ultrasound transducer probe 14. Transducer probe control module 30, as discussed in greater detail below with reference to FIG. 2, includes a beamformer that keeps ultrasound transducer probe 14 aimed at the renal blood flow of kidney 42L so that ultrasound transducer probe 14 continuously senses and communicates the Doppler flow signal DF of the renal blood flow to renal blood flow monitor 12 throughout the surgery, medical procedure, or medical observation of patient 10. Software code 22 can also include injury monitoring module 32 which includes acute kidney injury (AKI) monitoring software code and/or specific organ injury (SOI) monitoring software code. This code is monitoring software code that allows injury monitoring module 32 to determine, in real time, a characteristic of the renal blood flow of patient 10, monitor the characteristic of the renal blood flow over time, and determine an AKI risk score of patient 10 from the characteristic and the Doppler flow signal DF of the renal blood flow of kidney 42L. The AKI risk score represents the probability that kidney 42L is experiencing or approaching an AKI. When monitoring system 11 is used to monitor an organ other than kidneys 42L and 42R of patient 10, injury monitoring module 32 can be adapted to determine a real-time organ injury risk score from the Doppler flow signal of the organ blood flow of the organ that is being monitored, such as liver 44.

System processor 18 is a hardware processor configured to execute software code 22, which implements transducer probe control module 30 and injury monitoring module 32, to continuously sense the Doppler flow signal DF and monitor the Doppler flow signal for AKI of kidney 42L. Examples of system processor 18 can include any one or more of a microprocessor, a controller, a digital signal processor (DSP), an application specific integrated circuit (ASIC), a field-programmable gate array (FPGA), or other equivalent discrete or integrated logic circuitry.

Display 28 provides user interface 34, which includes control elements that enable user interaction with renal blood flow monitor 12 and/or other components of monitoring system 11. Display 28 is in communication with system processor 18 and is configured to provide plot 36 in real time of the Doppler flow signal DF of the renal blood flow of kidney 42L. In addition to showing plot 36 of Doppler flow signal DF, display 28 can also provide an audible representation of Doppler flow signal DF via a speaker. Display 28, as shown in FIG. 1, also shows an injury score indicator 38, which is a representation of the real-time AKI risk score of patient 10 determined from the Doppler flow signal DF by system processor 18 and injury monitoring module 32. Display 28 can also include a sensory alarm to alert medical personnel when the real-time AKI risk score of patient 10 is approaching or exceeding a predetermined threshold. The sensory alarm can be implemented as one or more of a visual alarm, an audible alarm, a haptic alarm, or other type of sensory alarm. For instance, the sensory alarm can be invoked as any combination of flashing and/or colored graphics shown by user interface 34 on display 28, a warning sound such as a siren or repeated tone, and a haptic alarm configured to cause renal blood flow monitor 12 to vibrate or otherwise deliver a physical impulse perceptible to medical personnel.

Display 28 can be a liquid crystal display (LCD), a light-emitting diode (LED) display, an organic light-emitting diode (OLED) display, or other display device suitable for providing information to users in graphical form. User interface 34 can include graphical and/or physical control elements that enable user input to interact with renal blood flow monitor 12 and/or other components of monitoring system 11. In some examples, user interface 34 can take the form of a graphical user interface (GUI) that presents graphical control elements presented at, e.g., a touch-sensitive and/or presence sensitive display screen of display 28. In such examples, user input can be received in the form of gesture input, such as touch gestures, scroll gestures, zoom gestures, or other gesture input. In certain examples, user interface 34 can take the form of and/or include physical control elements, such as a physical buttons, keys, knobs, or other physical control elements configured to receive user input to interact with components of monitoring system 11. User interface 34 can include a speaker that allows renal blood flow monitor 12 the ability to generate an audible alarm.

In operation of monitoring system 11, before a surgery, medical procedure, or medical observation begins, a medical worker places ultrasound transducer probe 14 on abdomen 40 of patient 10. The medical worker uses ultrasound transducer probe 14 to locate the Doppler flow signal DF of the renal blood flow of kidney 42L. Ultrasound transducer probe 14 can generate an audible representation of the Doppler flow signal DF to assist the medical worker in locating the Doppler flow signal DF of the renal blood flow of kidney 42L. Once the medical worker finds the Doppler flow signal DF of the renal blood flow of kidney 42L, the medical worker attaches and secures ultrasound transducer probe 14 to patient 10 with adhesive patch 16. Adhesive patch 16 keeps ultrasound transducer probe 14 in constant contact with patient 10 such that ultrasound transducer probe 14 does not shift positions on patient 10 during the surgery, medical procedure, or medical observation and lose the Doppler flow signal DF of the renal blood flow of kidney 42L. Ultrasound transducer probe 14 relays the received ultrasound signals to renal blood flow monitor 12 via cable(s) 24 or wirelessly. In the case of wireless transmission, the ultrasound transducer probe 14 includes the ultrasound front-end circuitry 17. System processor 18 of renal blood flow monitor 12 receives the Doppler flow signal DF and processes the Doppler flow signal DF sequentially or simultaneously through transducer probe control module 30 and injury monitoring module 32.

System processor 18 can execute the AKI monitoring software code of injury monitoring module 32 to establish a baseline value for the renal blood flow of kidney 42L of patient 10 from the Doppler flow signal DF sensed by ultrasound transducer probe 14. Deviations from the baseline value for the renal blood flow can be used as factors by system processor 18 and injury monitoring module 32 to calculate the real-time AKI risk score of kidney 42L. System processor 18 can further execute the AKI monitoring software code of injury monitoring module 32 to continuously monitor the Doppler flow signal DF of the renal blood flow sensed by ultrasound transducer probe 14 throughout a duration of the surgery, medical procedure, or medical observation of patient 10 and estimates the AKI risk score of kidney 42L of patient 10 from the Doppler flow signal DF. System processor 18 outputs the Doppler flow signal DF and the real-time AKI risk score of kidney 42L to display 28. Display 28 produces plot 36 showing the Doppler flow signal DF of the renal blood flow of kidney 42L plotted over time. Display 28 also produces injury score indicator 38 which represents the real-time AKI risk score of kidney 42L in injury score indicator 38.

As the surgery, medical procedure, or medical observation of patient 10 progresses, system processor 18 continues to receive the Doppler flow signal DF from ultrasound transducer probe 14 and continues to output both the Doppler flow signal DF and the real-time AKI risk score of kidney 42L to display 28. If the real-time AKI risk score of kidney 42L changes toward an undesired threshold, or changes at an undesired rate, system processor 18 and display 28 can alert the medical personnel so that the medical personnel can possibly take action to increase kidney perfusion and prevent AKI to kidney 42L, or minimize AKI to kidney 42L. For example, medical personnel can administer medication or fluids that increases the renal blood flow and perfusion to kidney 42L or improves autoregulation of the renal blood flow to kidney 42L. At the end of the surgery, medical procedure, or medical observation, system processor 18 and injury monitoring module 32 can estimate a final AKI risk score for kidney 42L and output the final AKI risk score to display 28. If the final AKI risk score for kidney 42L indicates that kidney 42L has a high risk of AKI, medical personnel can take immediate action to treat kidney 42L without having to wait for biomarkers to appear in blood and urine samples of patient 10. Biomarkers that indicate AKI can take several hours or days to appear in blood and urine samples of patient 10. With monitoring system 11, the medical personnel can determine quickly whether patient 10 needs to be treated for AKI of kidney 42L.

If kidney 42L of patient 10 moves within abdomen 40 of patient 10 during the surgery, medical procedure, or medical observation, transducer probe control module 30 will detect a change in the Doppler flow signal DF and will respond by adjusting the focusing location of the set of beams to scan abdomen 40 of patient 10 to relocate the Doppler flow signal DF and aim ultrasound transducer probe 14 at the new location of the Doppler flow signal DF of the renal blood flow of kidney 42L. As discussed below with reference to FIGS. 2-5, renal blood flow monitor 12 can include a beamformer that can steer beam signals produced by an array of transducer elements of ultrasound transducer probe 14.

FIG. 2 is another schematic diagram of renal blood flow monitor 12. As shown in FIG. 2, renal blood flow monitor 12 can include beamformer 48 with predictive filter 49. Ultrasound transducer probe 14 can include array 50 of transducer elements 52. Each transducer element 52 of array 50 can comprise a piezoelectric material, such as lead zirconate titanate, capable of transmitting ultrasound pulses and detecting ultrasound pulses. Array 50 of transducer elements 52 of ultrasound transducer probe 14 can form a two-dimensional phased array with probe length PL and probe width PW. As a phased array, each transducer element 52 in array 50 can pulse individually relative the other transducer elements 52 in array 50. Monitoring system 11 can also include breathing monitor 51, or can be in communication with breathing monitor 51.

In the example of FIG. 2, beamformer 48 drives array 50 of transducer elements 52 via system processor 18 and ultrasound front-end circuitry 17. Beamformer 48 functions as a transducer probe controller with flow signal tracking software code that controls the timing that each transducer element 52 in array 50 emits an ultrasound pulse. Beamformer 48 can time and pattern when each transducer element 52 emits a pulse such that array 50 can form one or more ultrasonic beams and can sweep or steer the one or more ultrasonic beams without physically moving the position of ultrasound transducer probe 14 on patient 10. Beamformer 48 can be a software sub-module of transformer probe control module 30 that can be executed by system processor 18 to control activation of transducer elements 52 of array 50. Predictive filter 49 can be a software sub-module of beamformer 48 and/or transformer probe control module 30 that can be executed by system processor 18 to predict an expected trajectory of a target vessel based on measured inputs from beamformer 48 and/or from inputs from other external sensors, such as breathing monitor 51. In other examples, beamformer 48 can be a separate hardware component from system processor 18 and system memory 20 with separate memory and software from software code 22 that coordinates with system processor 18 to control activation of transducer elements 52 of array 50. In the example of FIG. 2, beamformer 48 is housed within renal blood flow monitor 12 as part of transducer probe control module 30 of software code 22 that is executed by system processor 18. In other examples, beamformer 48 can be fully or partially housed within a casing of ultrasound transducer probe 14 as a separate hardware and software unit that coordinates with system processor 18. Housing beamformer 48 in the same unit as renal blood flow monitor 12 (whether as part of software code 22 or as an add-on hardware component) can decrease the overall size and thickness of ultrasound transducer probe 14. Ultrasound transducer probe 14 can be relatively thin and flat in profile, with a thickness that is smaller than a width or diameter of ultrasound transducer probe 14. Attaching ultrasound transducer probe 14 to patient 10 by adhesive patch 30 is easier and more secure when ultrasound transducer probe 14 has a thin and flat profile.

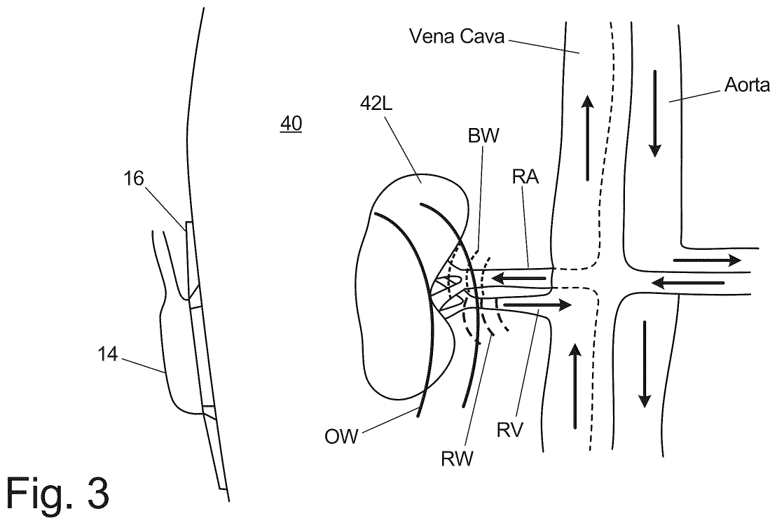

FIG. 3 is another schematic diagram of ultrasound transducer probe 14 attached to abdomen 40 of patient 10 by adhesive patch 16 over kidney 42L. The Doppler flow signal DF of kidney 42L can be measured from either the renal artery RA as blood enters kidney 42L from the aorta of patient 10 via the renal artery or from the renal vein RV as blood exits kidney 42L to the vena cava of patient 10 via the renal vein RV. Ultrasound transducer probe 14 generates originating signals OW that move into abdomen 40 of patient 10. Due to Doppler physics, a Doppler signal BW of the blood flow in the renal artery RA is “blue shifted” as the blood flow in the renal artery RA is moving toward the ultrasound transducer probe 14. A Doppler signal RW of the blood flow in the renal vein RV is “red shifted” as the blood flow in the renal vein RV is moving away from the ultrasound transducer. Since the Doppler signal BW is blue shifted and the Doppler signal RW is red shifted, renal blood flow monitor 12 can easily distinguish renal artery blood flow from renal vein blood flow. In human subjects the renal artery RA and renal vein RV are close and aligned parallel such that beamformer 48 can position the beam(s) to capture both arterial and venous flow of kidney 42L simultaneously.

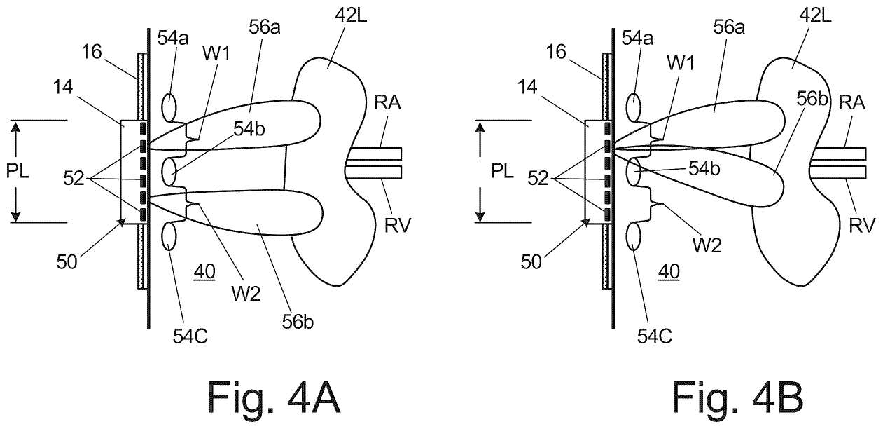

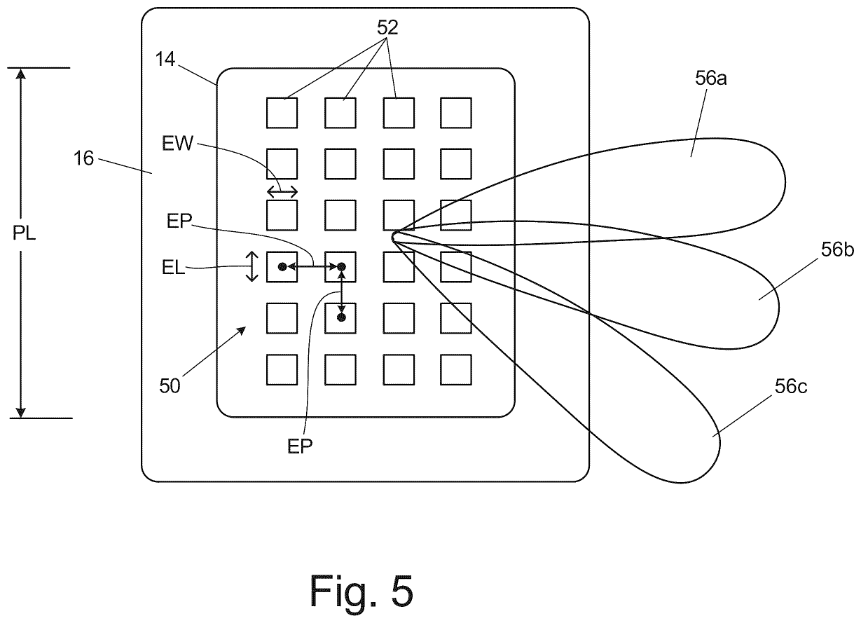

FIGS. 4A-5 will be discussed concurrently. FIG. 4A is another schematic diagram of ultrasound transducer probe 14 attached to abdomen 40 of patient 10 by adhesive patch 16 over kidney 42L. FIG. 4B is also a schematic diagram of ultrasound transducer probe 14 attached to abdomen 40 of patient 10 by adhesive patch 16 over kidney 42L. FIG. 5 is another schematic diagram of ultrasound transducer probe 14. In the example of FIGS. 4A and 4B, ultrasound transducer probe 14 is attached by adhesive patch 16 to a surface of abdomen 40 over kidney 42L and over at least some of ribs 54a, 54b, and 54c of patient 10.

Ultrasound transducer probe 14 can include a probe length PL, probe width PW (shown in FIG. 2), or diameter that is large enough that array 50 of transducer elements 52 of ultrasound transducer probe 14 can cover one or more acoustic windows in patient 10. An acoustic window of patient 10 is defined as an area of patient 10 where transmission of ultrasonic waves is not substantially attenuated in comparison to immediate surroundings. For example, array 50 of transducer elements 52 of ultrasound transducer probe 14 can be sized in length or width to extend over at least two intercostal spaces of patient 10. For example, in FIG. 4A, array 50 of transducer elements 52 of ultrasound transducer probe 14 is positioned over first acoustic window W1 (formed by the intercostal space between rib 54a and rib 54b) and over second acoustic window W2 (formed by the intercostal space between rib 54b and rib 54c). In the example of FIG. 4A, beamformer 48 (shown in FIG. 2) can selectively activate transducer elements 52 in array 50 to steer signal beams 56a, 56b, and 56c (not visible) into abdomen 40 through the first acoustic window W1 and/or second acoustic window W2 to avoid ribs 54a, 54b, and 54c. In the example of FIG. 4B, ultrasound transducer probe 14 is positioned slightly higher on abdomen 40 of patient 10 in comparison to the example of FIG. 4A. However, the probe length PL or probe width PW of ultrasound transducer probe 14 is long enough that ultrasound transducer probe 14 still has access to first acoustic window W1 and can still scan and steer signal beams 56a, 56b, and 56c (not visible) into abdomen 40 through the first acoustic window W1. Regardless of where ultrasound transducer probe 14 is placed over ribs 54a, 54b, and 54c, ribs 54a, 54b, and 54c will not block the direct view of kidney 42L from array 50 of ultrasound transducer probe 14.

Beamformer 48 controls transducer elements 52 in array 50 to beam scan abdomen 40 to locate a target vessel when ultrasound transducer probe 14 is first placed on patient 10. To find the target vessel, beamformer 48 divides the entirety of the field of view of array 50 into multiple sub-volumes and uses a predefined set of beams (such as beams 56a, 56b, and 56c) to probe each sub-volume. To reduce the search time, the search can be performed in two steps. In a first step the sub-volumes can be made larger in a depth dimension into abdomen 40 while a two-dimension scan is performed in the other two dimensions only. Once the location of the signal in the other two dimensions is determined by the two-dimension scan, the next step is to reduce the size of the sub-volume in the depth dimension and perform a search along the depth dimension at the previously determined location in the other two dimensions.

Beamformer 48 also controls transducer elements 52 in array 50 to track scan abdomen 40 to track the target vessel over time. Beamformer 48 beam scans and/or track scans the Doppler flow signal DF of the renal blood flow of kidney 42L of patient 10 by sequentially emitting signal beams 56a, 56b, and 56c from array 50 of transducer elements 52 and focusing each of beams 56a, 56b, and 56c in different locations. To track in both the azimuth dimension and the elevation dimension (sometimes referred to as altitude dimension), at least three beams are required. Using more beams will result in more accurate target vessel position estimation at the cost of a lower Nyquist frequency for the Doppler shift and hence the possibility of aliasing of the instantaneous spectrogram. Thus, beamformer 48 is not limited to three beams and can include more than three beams. The beam locations of beams 56a, 56b, and 56c are selected to have a sufficient degree of overlap of beams 56a, 56b, and 56c, such that when a target vessel is located at center of the three beams the signal-to-noise ratio of the Doppler flow signal in each of the beams is acceptably large (e.g. >20 dB). For example, the beam locations may be selected so that the center of beams 56a, 56b, and 56c lies at a point where the pressure is 3 dB below its peak value for each of beams 56a, 56b, and 56c.

By comparing integrated spectral power measured along multiple signal beams (e.g. beams 56a, 56b, and 56c), beamformer 48 and/or renal blood flow monitor 12 can estimate a bearing (i.e. the azimuthal and elevation angles) of the target vessel relative to array 50 of transducer elements 52. As a target vessel (e.g., the renal artery RA, and/or the renal vein RV) moves within abdomen 40, the target vessel will move closer to the focus of some of signal beams 56a, 56b, and 56c, which increases the integrated spectral power measured along those beams, and will move further away from the focus of some other(s) of signal beams 56a, 56b, and 56c, which decreases the integrated spectral power measured along those beams. As the target vessel moves, beamformer 48 can redirect signal beams 56a, 56b, and 56c (and possibly more signal beams) in the direction of those beams for which the measured integrated power spectrum is higher and away from those beams for which the integrated power spectrum is lower, thereby tracking the target vessel whose scatterers generate the Doppler flow signal DF. In one embodiment incorporating this tracking methodology, beamformer 48 computes an estimated location for the target vessel as a vector sum of unit vectors along the signal beam directions weighted by the integrated spectral power measured along each of signal beams 56a, 56b, and 56c. The weighting by the integrated spectral power ensures that as beamformer 48 redirects signal beams 56a, 56b, and 56c to the estimated target vessel location, the centroid of the beams 56a, 56b, and 56c will move towards those beams that have the largest integrated spectral power and therefore lie closest to the target vessel.

In another embodiment, beamformer 48 and/or renal blood flow monitor 12 can include a physical model that predicts the integrated spectral power for a given displacement between a signal beam and a target vessel to improve the estimate of the target vessel location. The model may, for example, calculate the integrated power spectrum as an overlap integral between an assumed beam shape (such as a Gaussian beam, a beam described by a sombrero function, or a beam described by a cardinal sine function, depending on transducer shape and apodization) and an assumed geometry for a target vessel such as a cylindrical vessel with a uniform density of moving scatterers across its cross-section. In some embodiments, the model may incorporate information about the change in beam shape with distance from transducer elements 52 as obtained from empirical measurements or acoustic simulation. In some embodiments the model may use an asymmetric beam shape such as an elliptical Gaussian beam with a narrower dimension and a wider dimension as would be produced by an asymmetric array of transducer elements. To estimate the target vessel location from the integrated power spectrum observed along multiple signal beams, the model is inverted using a standard function inversion methodology such as least-squares fitting, interpolation, series expansion, look-up tables and root-finding methods. Once the inverse function has been approximated, it can be used to obtain an estimate of the vessel target from the integrated spectral power measured along the signal beams.

In some embodiments, beamformer 48 and/or renal blood flow monitor 12 can use estimates of the target vessel location as an input to predictive filter 49, shown in FIG. 2, that contains a model of the expected trajectory of the target vessel. For example, in cases where the main source of target vessel motion is from breathing, predictive filter 49 may contain a periodic trajectory model describing the motion as periodic at the breathing frequency. In some embodiments, the periodic trajectory model may be implemented as a partial Fourier sum in each direction with the breathing frequency as the fundamental frequency. In such embodiments, model parameters may include some or all of the amplitude and phase (or equivalently, the amplitudes of the in-phase and quadrature components) of each Fourier component in each direction and the location of the origin about which the periodic motion occurs. In some embodiments, predictive filter 49 allows the model parameters to be updated in response to a target vessel position estimate obtained from the integrated power spectrum along a plurality of signal beams so that drift in the model parameters over time or the failure of the model to fully describe the trajectory may be accommodated.

In some embodiments, predictive filter 49 may incorporate an estimate of uncertainty in the estimate of the target vessel position obtained from the integrated power spectrum measurements. This uncertainty estimate may be used to adjust the degree to which the model parameters are affected by new measurements during parameter updates. In some embodiments, this uncertainty estimate may be used to force monitoring system 11 to ignore measurements that are invalid, due, for example, to a transient event that corrupts measurements over a period of time. In some embodiments, this uncertainty estimate may be used to reduce the degree to which measurements affect model parameters when the signal-to-noise ratio of the integrated power spectrum is low and to increase the degree to which measurements affect model parameters when the integrated power spectrum signal-to-noise ratio is high. In some embodiments, the uncertainty estimate may be adjusted in response to changes in the moments of the instantaneous spectrum of the Doppler flow signal (e.g. the mean velocity, the spectral bandwidth), or the maximum velocity envelope of the Doppler spectrogram. In some embodiments, the uncertainty estimate may be adjusted based on the total integrated power spectrum, including both the negative and positive frequencies, or based on an integrated power spectrum in a different range of Doppler shifts than the range used to estimate target vessel position. For example, the integrated power spectrum over the negative frequencies may be used to estimate the uncertainty in a position estimate arrived at using the integrated power spectrum over the positive frequencies.

The integrated spectral power is an inherently noisy signal as the Doppler spectrogram contains speckle arising from constructive and destructive interference between large numbers of scatterers distributed randomly through the insonified volume of abdomen 40 and from statistical noise due to variance in the number and orientation of scatterers in the beam(s) over time. Additionally, the integrated power spectrum is modulated by the cardiac cycle because a larger fraction of scatterers will have Doppler shifts large enough to pass through the filter that defines the Doppler flow signal during systole than during diastole. If unmitigated, the variability in the integrated power spectrum due to speckle and the cardiac cycle will lead to a noisy estimate of target vessel location and to inaccurate tracking. In some embodiments, the noise on the integrated power spectrum is reduced by applying a filter to the integrated power spectrum signal prior to using the integrated power spectrum signal to estimate the target vessel location. Making a kernel duration of the filter longer will make the filter more effective at removing noise, but if the kernel duration of the filter becomes comparable to a timescale of target vessel motion of the target vessel, then the filter will begin to degrade tracking accuracy. Since the fastest source of the target vessel motion is breathing, a filter kernel size shorter than the breathing cycle duration advantageously reduces modulation from cardiac cycle and speckle when maintaining target vessel location estimation accuracy. Statistical noise and speckle noise produce long-tailed intensity distributions with a high probability of producing very large values. Consequently, because of these occasional very large intensities, linear filters are ineffective at smoothing the integrated power spectrum. Median filters are advantageously insensitive to outliers and provide a smoother output than is possible with linear filters. Consequently, in some embodiments, a median filter is used to filter the integrated power spectrum. In some embodiments the median filter kernel size is selected to be larger than the cardiac cycle duration but less than the breathing cycle duration.

In some embodiments, information obtained from other sensors separate from ultrasound transducer probe 14 or a priori information may also be provided to predictive filter 49 estimating the target vessel location. Predictive filter 49 may be configured to incorporate this additional information when adjusting the model parameters as well as adjusting the estimate of target vessel position obtained from the integrated power spectrum. For example, predictive filter 49 may receive input from breathing monitor 51 connected to patient 10 and may use measurements from breathing monitor 51 to update model parameters that capture a breathing frequency of patient 10. In some embodiments, predictive filter 49 can incorporate both measurements made with external sensors (such as breathing monitor 51) and the estimate of target vessel position obtained from the integrated power spectrum to adjust model parameters. In some embodiments, predictive filter 49 may use information obtained from integrated power spectrum measurements taken at an earlier point in time. For example, in some embodiments, tracking of the target vessel may be halted and the directions of signal beams 56a, 56b, and 56c may be fixed in order to observe the periodicity in the integrated power spectrum as the target vessel moves due to breathing. This observation may be used to estimate breathing frequency so that the breathing frequency may be incorporated into predictive model 49 when tracking resumes.

In some embodiments, predictive filter 49 is implemented as a Linear Kalman Filter. In some embodiments, predictive filter 49 is implemented as an Unscented Kalman Filter. In some embodiments, predictive filter 49 is implemented as an Extended Kalman Filter.

In some embodiments, predictive filter 49 may be configured to produce an estimate of the integrated power spectrum signal along each of a plurality of signal beams (such as signal beams 56a, 56b, and 56c) based on an internal parametric model of target vessel position, beam shape and target vessel shape and orientation. The estimate of the integrated power spectrum by predictive filter 49 for each of the plurality of signal beams may be compared to measurements of the integrated power spectrum along each signal beam, and the difference between the prediction and measurement can be used to update the model parameters including those describing the target vessel location. In calculating the integrated power spectrum along the plurality of signal beams, predictive filter 49 may make use of a physical model of the integrated power spectrum that calculates an overlap integral between the target vessel and the ultrasound beam profile. In some embodiments the physical model may include a description of how the beam profile changes with depth. In some embodiments the physical model may include an asymmetric beam profile such as would be produced by an asymmetric transducer array.

In some embodiments, differences in integrated power spectrum between the different signal beams 56a, 56b, and 56c are used by beamformer 48 and/or renal blood flow monitor 12 to estimate the bearing (azimuthal and elevation angles) of the target vessel, while the range (distance from the transducer) of the target vessel is estimated by beamformer 48 and/or renal blood flow monitor 12 by calculating the integrated power spectrum at a plurality of range samples, assigning a likelihood of containing the target vessel to each range sample, and calculating an estimate of the center of the target vessel from the plurality of range samples. In some embodiments, the likelihood that a range sample contains the target vessel is made proportional to the integrated power spectrum at that range so that the estimate of the location of the target vessel range may be estimated, for example, by beamformer 48 and/or renal blood flow monitor 12 by selecting the range sample with the largest integrated power spectrum or calculating the location of the centroid over the range samples. In other embodiments, beamformer 48 and/or renal blood flow monitor 12 can use a likelihood function to take into account integrated power spectrum, spectral moments, Doppler spectrogram shape, and/or integrated power in spectral ranges other than the range where the integrated power spectrum is calculated. In many cases, the target vessel may extend over a plurality of range samples, in which case, the accuracy of the integrated power spectrum may be improved by averaging over the plurality of range samples likely to contain the target vessel.

In some embodiments, an estimate of target vessel range incorporates the integrated power spectrum calculated for each of a plurality of signal beams (e.g. 56a, 56b, 56c). In some embodiments, beamformer 48 and/or renal blood flow monitor 12 can arrive at this estimate by first averaging the integrated power spectrum across the plurality of beams at each range sample and then calculating a likelihood of each range sample containing the target on this averaged signal.

Separating the estimate of the bearing of the target vessel from the estimate of the range of the target vessel in this way is advantageous as the beamformer 48 and/or renal blood flow monitor 12 can calculate the range estimations more frequently than the bearing estimations over time. Beamformer 48 and/or renal blood flow monitor 12 can obtain a range estimate on every ultrasound transmit event, while a bearing estimate requires that beamformer 48 move an ultrasound beam to a plurality of locations and that the measurements made at the different locations be compared by beamformer 48 and/or renal blood flow monitor 12. Having a reliable estimate of range associated with each transmit event ensures that when beamformer 48 and/or renal blood flow monitor 12 uses the integrated power spectrum to estimate bearing across a plurality of signal beams, the integrated power spectrum from the range or ranges closest to the target vessel are used by beamformer 48 and/or renal blood flow monitor 12 in the bearing calculation. The separation of range from bearing estimation also simplifies the predictive model used to estimate bearing thereby making the predictive model more robust and reliable.

Similarly, when beamformer 48 and ultrasound transducer probe 14 scans across the field of view to locate the target vessel, the separation of range estimation from bearing estimation advantageously reduces the number of dimensions over which beamformer 48 and ultrasound transducer probe 14 must scan the beam from three dimensions to two dimensions. The estimation methods described in the preceding paragraphs apply equally well to scanning as to tracking.

In order for ultrasound transducer probe 14 to measure the Doppler flow signal DF of the renal blood flow of kidney 42L, ultrasound transducer probe 14 can have a low center frequency between 0.5 MHz and 4.0 MHz. With a center frequency between 0.5 MHz and 4.0 MHz, ultrasound transducer probe 14 can penetrate more than 15 cm into patient 10, which is a sufficient depth to measure the renal blood flow. This depth also allows ultrasound transducer probe 14 the ability to measure hepatic blood flow, celiac blood flow, portal blood flow, and mesenteric blood flow.

As shown best in the example of FIG. 5, each transducer element 52 in array 50 comprises an element width EW and element length EL that are both larger than one wavelength in soft tissue of an ultrasonic wave emitted by array 50 of transducer elements 52. Array 50 of transducer elements 52 also includes a pitch EP defining an inter-element spacing between centers of adjacent transducer elements 52. In the example of FIG. 5, the pitch EP is larger than the one wavelength in soft tissue of the ultrasonic wave emitted by array 50 of transducer elements 52. The element width EW, the element length EL, and the pitch EP are all larger than the one wavelength in soft tissue of the ultrasonic wave emitted by array 50 of transducer elements 52 to reduce an element count for the selected aperture of ultrasound transducer probe 14. In a traditional phased array imaging transducer, use of a pitch of greater than one wavelength would result in significant image degradation due to grating lobes. However, for ultrasound transducer probe 14, grating lobes do not degrade the Doppler spectrogram because large blood vessels are sparsely distributed in the body and it is highly unlikely that an interfering Doppler signal source would be located at a grating lobe location when a main lobe is focused on a target vessel. Monitoring system 11 does not use ultrasound transducer probe 14 for high resolution imaging of kidney 42L, thus ultrasound transducer probe 14 does not need to have as high a transducer element count as an ultrasound transducer probe used for ultrasound imaging.

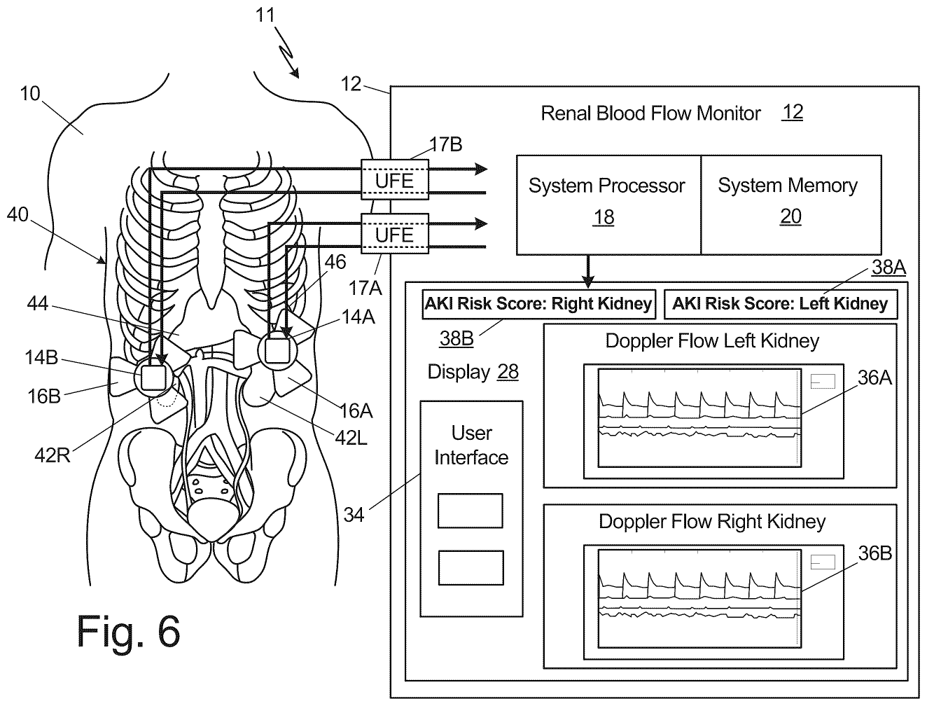

Multiple continuous organ blood flow sensors can be placed on a single patient. This can be advantageous for monitoring multiple organs at once, particularly both kidneys. FIG. 6 shows two ultrasound transducer probes 14A and 14B connected to patient 10 with a single renal blood flow monitor 12 connected to both ultrasound transducer probes 14A and 14B. Ultrasound transducer probe 14A is positioned over left kidney 42L to detect and track the renal blood flow of left kidney 42L. Ultrasound transducer probe 14B is positioned over right kidney 42R to detect and track the renal blood flow of right kidney 42R. Renal blood flow monitor 12 in FIG. 6 receives Doppler flow signals from both ultrasound transducer probes 14A and 14B and can output first plot 36A of a Doppler flow signal of the renal blood flow of left kidney 42L to display 28, and output second plot 36B of a Doppler flow signal of the renal blood flow of right kidney 42R to display 28. Renal blood flow monitor 12 in FIG. 6 can also output first injury score indicator 38A and second injury score indicator 38B to display 28. First injury score indicator 38A is a representation of the real-time AKI risk score of left kidney 42L and second injury score indicator 38B is a representation of the real-time AKI risk score of right kidney 42R.

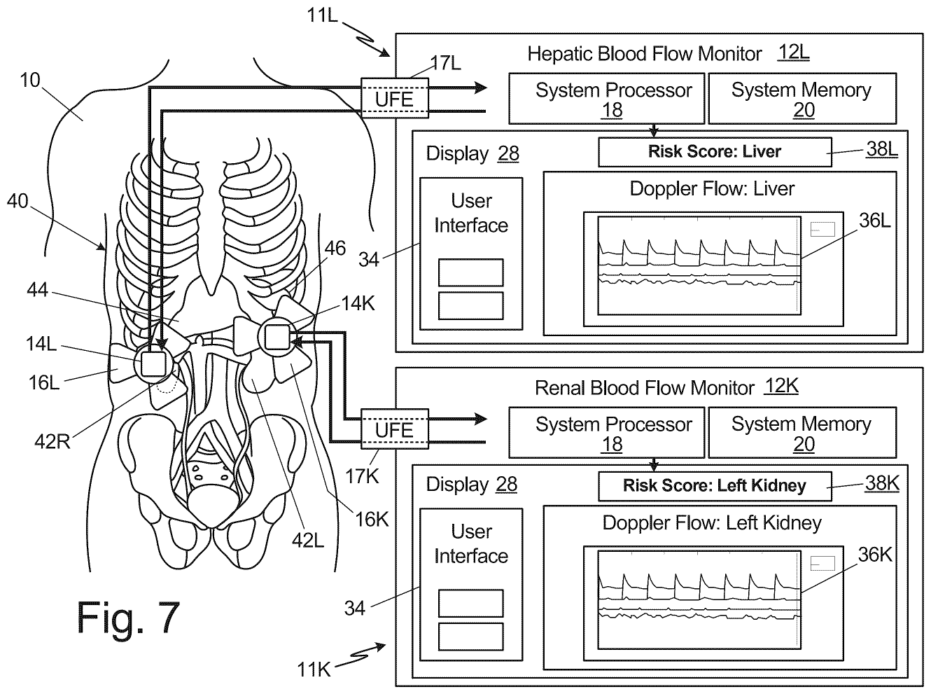

FIG. 7 is a schematic diagram of patient 10 with two monitoring systems 11K and 11L. Monitoring system 11K includes renal blood flow monitor 12K and ultrasound transducer probe 14K positioned on a left side of abdomen 40 of patient 10 to monitor the renal blood flow of left kidney 42L. Monitoring system 11K functions in a similar manner to monitoring system 11 described above with reference to FIGS. 1-6. Renal blood flow monitor 12K outputs plot 36K of a Doppler flow signal of the renal blood flow of left kidney 42L to display 28. Renal blood flow monitor 12K in FIG. 7 can also output injury score indicator 38K to display 28. Injury score indicator 38K is a representation of the real-time AKI risk score of left kidney 42L.

Monitoring system 11L includes hepatic blood flow monitor 12L and ultrasound transducer probe 14L positioned over a right side of abdomen 40 of patient 10 to monitor blood flow in the hepatic or portal veins of liver 44. Monitoring system 11L functions in a similar manner to monitoring system 11 described above with reference to FIGS. 1-6. Hepatic blood flow monitor 12L outputs plot 36L of a Doppler flow signal of the blood flow of liver 44 to display 28. Hepatic blood flow monitor 12L in FIG. 7 can also output injury score indicator 38L to display 28. Injury score indicator 38L is a representation of the real-time organ injury risk score of liver 44.

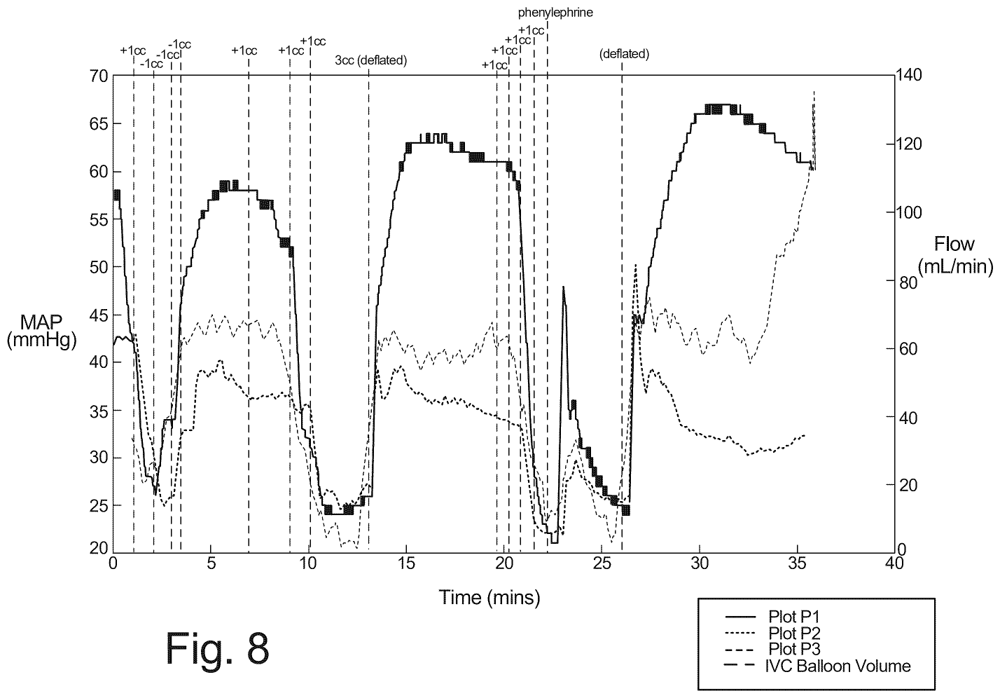

FIG. 8 is a chart from an experiment demonstrating monitoring system 11. The chart shows three plots. First plot P1 is a plot of mean arterial pressure (MAP) of a test subject (a pig) that was measured by a hemodynamic sensor over time. Second plot P2 is a plot of a renal blood flow of the test subject over time that was measured by an invasive flow probe that was surgically implanted around a renal artery of the test subject to provide a reference measurement of the renal blood flow. Third plot P3 is a plot of the renal blood flow index of the test subject as measured by ultrasound transducer probe 14 of monitoring system 11 over time. The experiment lasted at least forty minutes. A balloon catheter was inserted in the inferior vena cava of the test subject. During the experiment, the balloon catheter was inflated and deflated several times to cause decreases and increases in the MAP of the test subject, as represented in the chart by dashed vertical lines. When the balloon catheter was inflated, the MAP of the test subject would decrease, which also caused the renal blood flow of the test subject to decrease. When the balloon catheter was deflated, the MAP of the test subject would increase, which also caused the renal blood flow of the test subject to increase. As indicated in plots P2 and P3, non-invasive ultrasound transducer probe 14 of monitoring system 11 in this experiment was able to identify the changes in the renal blood flow of the test subject in a similar manner as the invasive transonic flow probe that was surgically implanted around the renal artery of the test subject.

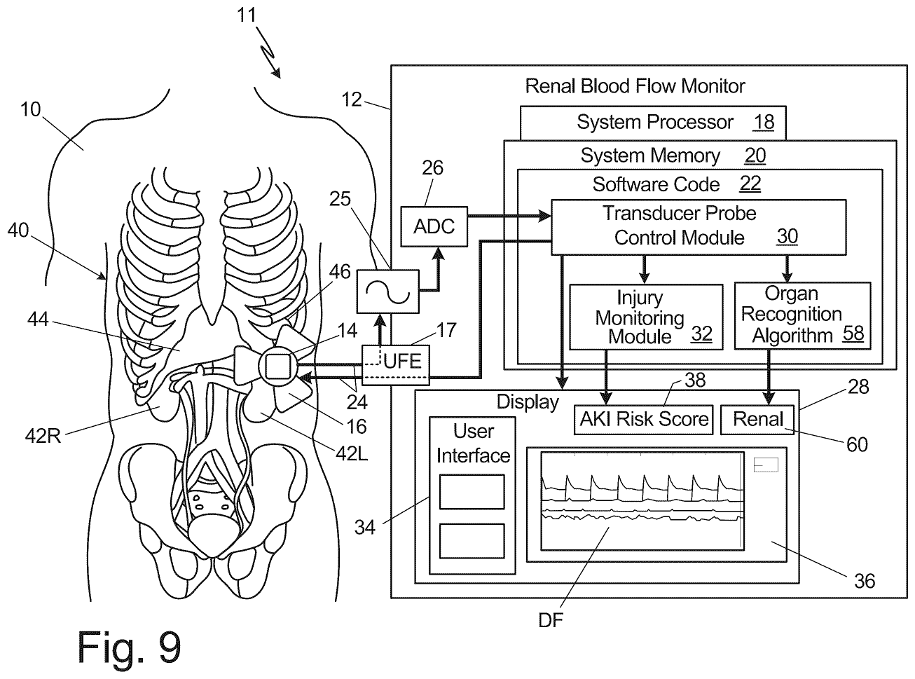

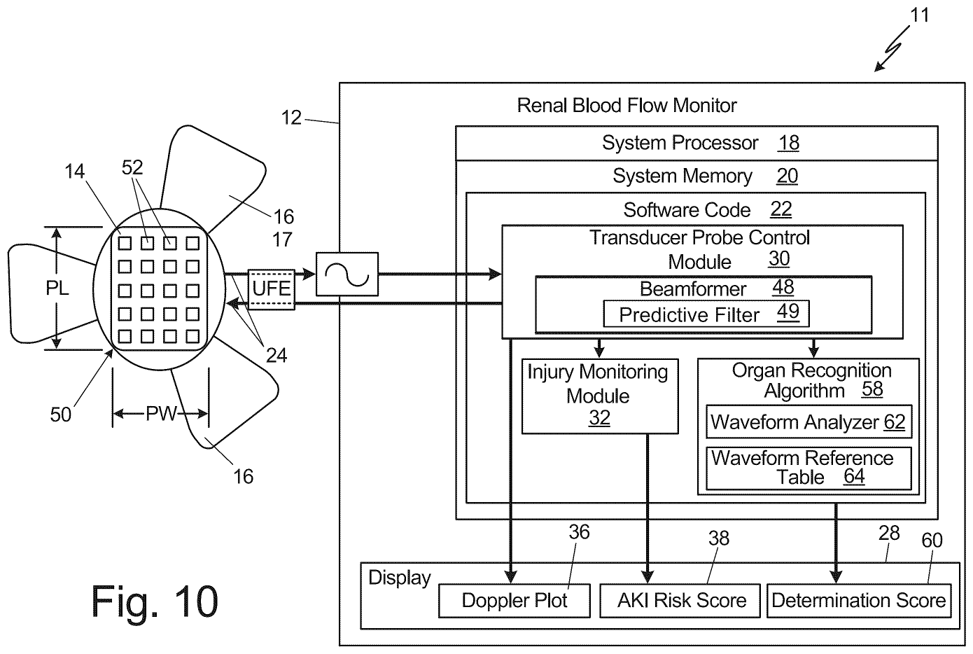

FIGS. 9 and 10 will be discussed concurrently. FIGS. 9 and 10 are schematic diagrams of monitoring system 11 of FIGS. 1 and 2 with the addition of organ recognition algorithm 58. In the example of FIGS. 9 and 10, organ recognition algorithm 58 is a software module stored in system memory 20 as part of software code 22. Organ recognition algorithm 58 can be based on either machine learning or standard signal processing. When executed by system processor 18, organ recognition algorithm 58 can recognize and distinguish a targeted organ blood flow signal of patient 10 from non-targeted blood flow signals based upon waveform characteristics of the targeted organ blood flow signal. In the example of FIGS. 9 and 10, the targeted organ blood flow signal is the Doppler flow signal DF of the renal blood flow of kidney 42L, and organ recognition algorithm 58 recognizes the Doppler flow signal DF of the renal blood flow from other non-renal blood flow signals based upon waveform characteristics of the Doppler flow signal DF. Organ recognition algorithm 58 can aid renal blood flow monitor 12 in monitoring the renal blood flow of kidney 42L by verifying that system processor 18 and transducer probe control module 30 are continually aiming ultrasound transducer probe 14 electronically at the Doppler flow signal DF of the renal blood flow and not mistakenly aiming at some other organ blood flow. This feature can be very useful as monitoring system 11 monitors patient 10 over time as kidney 42L and other organs can shift and move within abdomen 40, causing the Doppler flow signal DF to drift relative to ultrasound transducer probe 14 or cause other organ blood flow signals to appear within a sensing window of ultrasound transducer probe 14.

As shown in FIG. 10, organ recognition algorithm 58 can include waveform analyzer 62 and waveform reference table 64. Waveform reference table 64 is a table of renal blood flow waveform characteristics and non-renal blood flow waveform characteristics. For example, waveform reference table 64 can include a sub-table of waveform characteristics that belong to the Doppler flow signal DF of the renal blood flow of kidney 42L. Waveform reference table 64 can include another sub-table of waveform characteristics that belong to a Doppler signal of the hepatic blood flow of liver 44. Waveform reference table 64 can include another sub-table of waveform characteristics that belong to a Doppler signal of the portal blood flow of the stomach (not shown). Waveform reference table 64 can be prepopulated with waveforms obtained from prior measurements from a population. Waveform reference table 64 can also include information from patient 10 that is gathered in advance of the operation, medical procedure, or medical observation by scanning each organ of patient 10 in the region of abdomen 40 that will be monitored by monitoring system 11. For example, before monitoring system 11 is attached to patient 10 to monitor the kidney blood flow of kidney 42L, a technician can use ultrasound transducer probe 14 and renal blood flow monitor 12 to scan the region of abdomen 40 around kidney 42L and use waveform analyzer 62 to collect waveform characteristics of each significant blood flow signal in the region and populate waveform reference table 64 that is specific to patient 10. Once waveform reference table 64 has been populated, the technician can relocate the Doppler flow signal DF of the renal blood flow of kidney 42L with ultrasound transducer probe 14 and can attach ultrasound transducer probe 14 to patient 10 with adhesive patch 16.

Waveform analyzer 62, when executed by system processor 18, performs waveform analysis of the Doppler flow signal DF of the renal blood flow sensed by ultrasound transducer probe 14 and extracts waveform characteristics of the Doppler flow signal DF. Then, system processor 18 can execute waveform analyzer 62 to compare the waveform characteristics of the Doppler flow signal DF to waveform reference table 64 to verify that the Doppler flow signal DF is indeed the signal of the renal blood flow of kidney 42L. After comparing the waveform characteristics of the Doppler flow signal DF to waveform reference table 64, system processor 18 and waveform analyzer 62 outputs determination score 60, or a representation of determination score 60, to display 28. Determination score 60 indicates whether the Doppler flow signal DF is from the renal blood flow or from a non-renal blood flow. For example, as shown in FIG. 9, determination score 60 can state “Renal” when the Doppler flow signal DF is from the renal blood flow. If monitoring system 11 is being used to monitor hepatic flow of liver 44, determination score 60 can state “Hepatic” when monitoring system 11 finds a Doppler flow signal for the hepatic flow. In other examples, determination score 60 can use numerical indicators or acronyms. In yet another example, determination score 60 can include a quality grade or index of the signal of interest that is assessed from the waveform characteristics of the Doppler flow signal DF. Determination score 60 and the quality grade or index is continuously provided to the Kalman filter so the Kalman filter can more accurately estimate the reliability of the measurement in real-time and to properly estimate the future location based on a weighted balance between the predictive model and the current measurement.

The quality grade or index of determination score 60 distinguishes acceptable Doppler flow signals (DF) of blood flow from noise, artifacts, or other physiologically irrelevant blood flow signals. Waveform analyzer 62 establishes the quality grade or index embodied by the determination score by comparing Doppler flow signals DF with waveform reference table 64. In certain embodiments, system processor 18 can execute waveform analyzer 62 to calculate signal features such as the Signal-to-Noise Ratio (SNR), integrated power spectrum, spectral envelope, pulsatility, and spectral bandwidth.

The signal features calculated and collected by waveform analyzer 62 are then subjected to statistical processing to ensure classification of the outputs from beamformer 48. Such classifications might include identifying signals as ‘Renal’, ‘Hepatic’, arterial, venous, noise, artifact, among others. In some embodiments, this multi-class classification is generated by the application of a machine learning based classifier. The machine learning based classifier can be trained on labelled training datasets of Doppler flow signals that use outputs of the waveform analyzer 62 (for example, SNR, integrated power spectrum, spectral envelope, pulsatility, and spectral bandwidth) as input features. Waveform analyzer 62 can use various classifier models to classify Doppler flow signals including Random Forest Classifiers and Support Vector Machine (SVM) classifiers. One embodiment of this classification system was trained on a dataset of 5,756 samples, each sample including a 0.25s segment of a Doppler signal taken from one of four healthy subjects. The dataset was 17 minutes in total time, with some of the samples of the dataset overlapping one another. Each sample was labeled as either “renal arterial flow”, “non-blood artifact”, or “noise”. Following training, the model was able to correctly classify renal arterial flow with 93% accuracy on a training set of 2,467 samples.

In some embodiments, waveform analyzer 62 employs machine learning-based classifiers that not only provide a predicted class label but also provide an estimate of the confidence in the classification in terms of a probability of correct classification. For example, in embodiments of waveform analyzer 62 employing a Random Forest classifier model, the proportion of trees voting for a particular classification may be interpreted as a confidence score ranging from 0 to 1. In embodiments of waveform analyzer 62 employing an SVM classifier, each of the SVM scores gives the distance of a sample to decision hyperplanes in feature space. Waveform analyzer 62 can use a logistic regression model to convert these SVM scores into probabilities for various binary classifications. Waveform analyzer 62 can use any other machine learning or classification algorithm that can produce a probability of correct classification.

In some embodiments, the classification of Doppler flow signal DF and the probability of correct classification of Doppler flow flow signal DF are used to establish a measurement uncertainty that is passed into the Kalman filter being used by beamformer 48 and/or renal blood flow monitor 12 to track a target vessel location. In some embodiments the classifier is configured as a binary classifier that classifies Doppler flow signal into “blood flow from the target vessel” and “not blood flow from the target vessel” along with an estimate of the probability that the classification is correct. A function maps the probability range [0,1] of the Doppler flow signal being blood flow from the target vessel to an uncertainty range [∞,0] where an uncertainty of ∞ indicates complete certainty that the Doppler flow signal is not blood flow from the target vessel and 0 indicates complete certainty that the Doppler flow signal is blood flow from the target vessel. In various embodiments the function mapping probability of correct classification to measurement uncertainty may be a rational polynomial function, a logit, a logarithm, or an exponential function or another function that maps from [0,1] to [∞,0].

In some embodiments, classification and the probability of correct classification may be applied to the problem of searching for a target vessel such as a Renal or Hepatic vessel by assigning a probability to each scan of abdomen 40 by the array 50. The target vessel is then identified as being located at the location in the scan that is classified as containing flow from the target vessel with the highest probability of correct classification.

While the organ recognition algorithm 58 has been described as distinguishing the Doppler flow signal DF of the renal blood flow from non-renal blood flow signals of patient 10, organ recognition algorithm 58 can be used to identify other organ blood flow signals. For example, if monitoring system 11 is being used on patient 10 to monitor hepatic blood flow of liver 44, organ recognition algorithm 58 can be executed by system processor 18 to distinguish and verify a Doppler flow signal of the hepatic blood flow of liver 44 from the renal blood flow or other organ blood flow signals of patient 10.

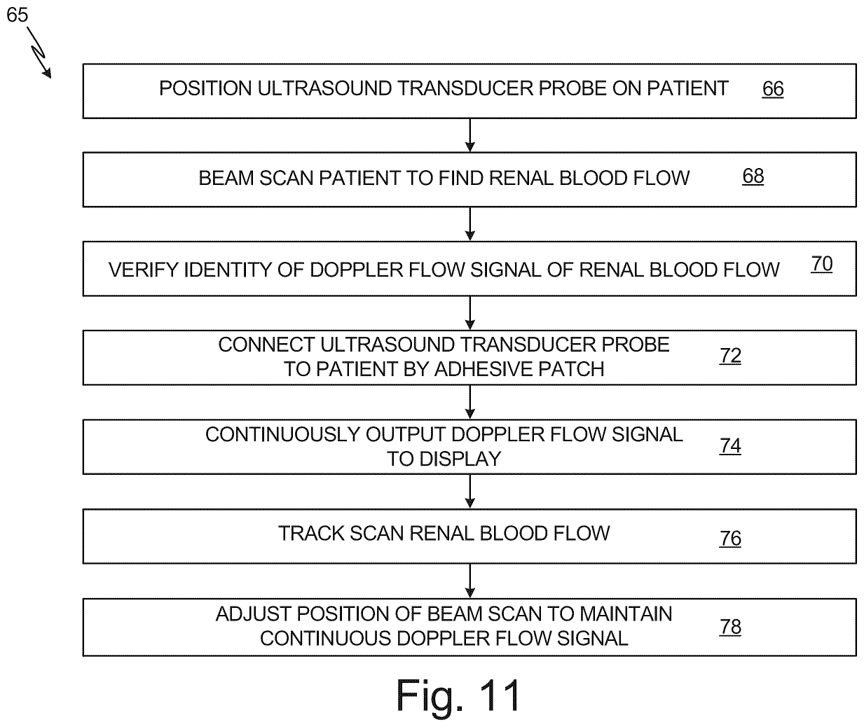

FIG. 11 is a block diagram of one method 65 for operating monitoring system 11 shown in FIGS. 9 and 10 to continuously monitor the renal blood flow and perfusion of kidney 42L of patient 10. First step 66 of method 65 includes positioning ultrasound transducer probe 14 on patient 10. Next, in second step 68, system processor 18 executes transducer probe control module 30 with beamformer 48 to beam scan patient 10 with ultrasound transducer probe 14 to find the Doppler flow signal DF of the renal blood flow of kidney 42L. If the Doppler flow signal DF is not found, renal blood flow monitor 12 can alert and instruct the medical personnel to reposition ultrasound transducer probe 14 on patient 10 and repeat second step 68. In third step 70, system processor 18 can optionally execute organ recognition algorithm 58 to verify that the Doppler flow signal DF is in fact the signal of the renal blood flow of kidney 42L. With the Doppler flow signal DF found and verified, fourth step 72 of method 65 can be performed by attaching and securing ultrasound transducer probe 14 to patient 10 by adhesive patch 16. Adhesive patch 16 keeps ultrasound transducer probe 14 in place on patient 10 and maintains contact between abdomen 40 and ultrasound transducer probe 14 so that monitoring system 11 can continue to sense and analyze the Doppler flow signal DF over an extended time period, such as a surgery or a stay in an intensive care unit (ICU) or an emergency ward. In fifth step 74 of method 65, system processor 18 continuously outputs a plot of the Doppler flow signal DF of the renal blood flow to display 28. As part of fifth step 74, system processor 18 executes injury monitoring module 32 to estimate a real-time AKI risk score of patient 10 from the Doppler flow signal DF and output a representation of the real-time AKI risk score to display 28 as injury score indicator 38. While renal blood flow monitor 12 continues to read the Doppler flow signal DF of the renal blood flow, system processor 18 performs sixth step 76 by executing transducer probe control module 30 to track scan the Doppler flow signal DF as described above with reference to FIGS. 2-5. If the Doppler flow signal DF of the renal blood flow begins to shift and drift relative ultrasound transducer probe 14, system processor 18 can perform seventh step 78 by executing transducer probe control module 30 to adjust a position or angle of a beam scan of ultrasound transducer probe 14 to follow the Doppler flow signal DF.

As the surgery or medical procedure of patient 10 progresses, or as the stay of patient 10 in the ICU or emergency ward passes in time, system processor 18 continues to receive the Doppler flow signal DF from ultrasound transducer probe 14 and continues to output both the Doppler flow signal DF and the real-time AKI risk score of kidney 42L to display 28. If the real-time AKI risk score of kidney 42L changes toward an undesired threshold, or changes at an undesired rate, system processor 18 and display 28 can alert medical personnel so that the medical personnel can possibly take action to increase kidney perfusion and prevent AKI to kidney 42L, or minimize AKI to kidney 42L. For example, medical personnel can administer medication or fluids that increases the renal blood flow and perfusion to kidney 42L or improves autoregulation of the renal blood flow to kidney 42L. Monitoring system 11 allows medical personnel to take immediate action to treat kidney 42L without having to wait for biomarkers to appear in blood and urine samples of patient 10. Biomarkers that indicate AKI can take several hours or days to appear in blood and urine samples of patient 10. With monitoring system 11, the medical personnel can determine quickly whether patient 10 needs to be treated for AKI of kidney 42L.

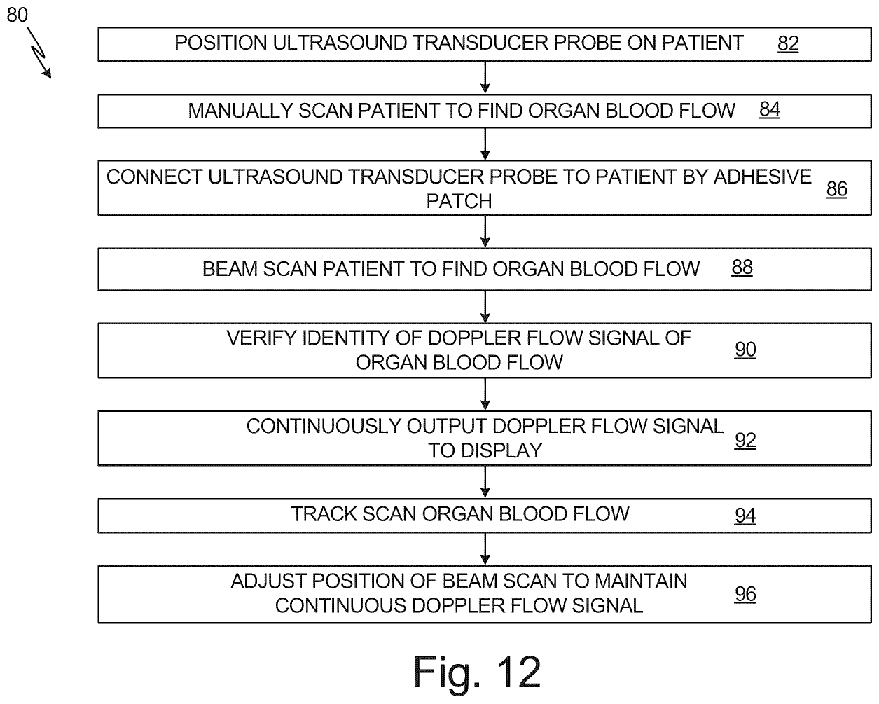

FIG. 12 is a block diagram of another method 80 for operating monitoring system 11 shown in FIGS. 9 and 10 to continuously monitor an organ blood flow of a targeted organ of patient 10 and help maintain proper perfusion of the targeted organ. The targeted organ of patient 10 can be kidney 42L or kidney 42R, or any other organ of patient 10, such as liver 44, spleen 46, the pancreas, the stomach, the intestines, the heart, and the brain. First step 82 of method 80 includes positioning ultrasound transducer probe 14 on patient 10. Next, in second step 84, a technician manually scans patient 10 with ultrasound transducer probe 14 of monitoring system 11 (or another ultrasound probe) to find an organ blood flow signal of the targeted organ. During second step 84, the technician can also populate waveform reference table 64 of organ recognition algorithm 58 by scanning the organ blood flow signal of the targeted organ and the organ blood flow signals of surrounding organs and feeding those signals through system processor 18 and waveform analyzer 62.

Once the organ blood flow signal of the targeted organ has been found, the technician performs third step 86 by attaching and securing ultrasound transducer probe 14 to patient 10 by adhesive patch 16 at the location where the organ blood flow signal was found in second step 84. Adhesive patch 16 keeps ultrasound transducer probe 14 in place on patient 10 and maintains contact between abdomen 40 and ultrasound transducer probe 14 so that monitoring system 11 can continue to sense and analyze the organ blood flow signal over an extended time period, such as a surgery or a stay in an ICU or an emergency department. With ultrasound transducer probe 14 secured to patient 10 by adhesive patch 16, system processor 18 can proceed with fourth step 88 by executing transducer probe control module 30 with beamformer 48 to command ultrasound transducer probe 14 to beam scan patient 10 to re-find the organ blood flow signal of the targeted organ. System processor 18 can also perform fifth step 90 to verify that the ultrasound transducer probe 14 is indeed sensing and reading the organ blood flow of the targeted organ and not aimed at an undesired flow signal.

With the organ blood flow signal of the targeted organ found and verified, system processor 18 performs sixth step 92 of method 80 by continuously outputting a plot of the organ blood flow signal of the targeted organ to display 28. As part of sixth step 92, system processor 18 can also execute injury monitoring module 32 to estimate a real-time organ injury risk score of patient 10 from the organ blood flow signal and output a representation of the real-time organ injury risk score to display 28 as injury score indicator 38. While blood flow monitor 12 continues to read and analyze the organ blood flow signal of the targeted organ, system processor 18 performs seventh step 94 by executing transducer probe control module 30 to track scan the organ blood flow signal of the targeted organ as described above with reference to FIGS. 2-5. If the organ blood flow signal of the targeted organ begins to shift and drift relative to ultrasound transducer probe 14, system processor 18 can perform eighth step 96 by executing transducer probe control module 30 to adjust a position or angle of a beam scan of ultrasound transducer probe 14 to follow the organ blood flow signal of the targeted organ.

As the surgery or medical procedure of patient 10 progresses, or as the stay of patient 10 in the ICU or emergency ward passes in time, system processor 18 continues to receive the organ blood flow signal of the targeted organ from ultrasound transducer probe 14 and continues to output both the plot of the organ blood flow signal and the real-time organ injury risk score of the targeted organ to display 28. If the real-time organ injury risk score of the targeted organ changes toward an undesired threshold, or changes at an undesired rate, system processor 18 and display 28 can alert medical personnel so that the medical personnel can possibly take action to increase perfusion (or decrease perfusion) to the targeted organ to prevent or minimize injury to the targeted organ. For example, medical personnel can administer medication or fluids that increases blood flow to the targeted organ or improves autoregulation of the targeted organ. Monitoring system 11 allows medical personnel to take immediate action to the targeted organ without having to wait for biomarkers to appear in blood and urine samples of patient 10.

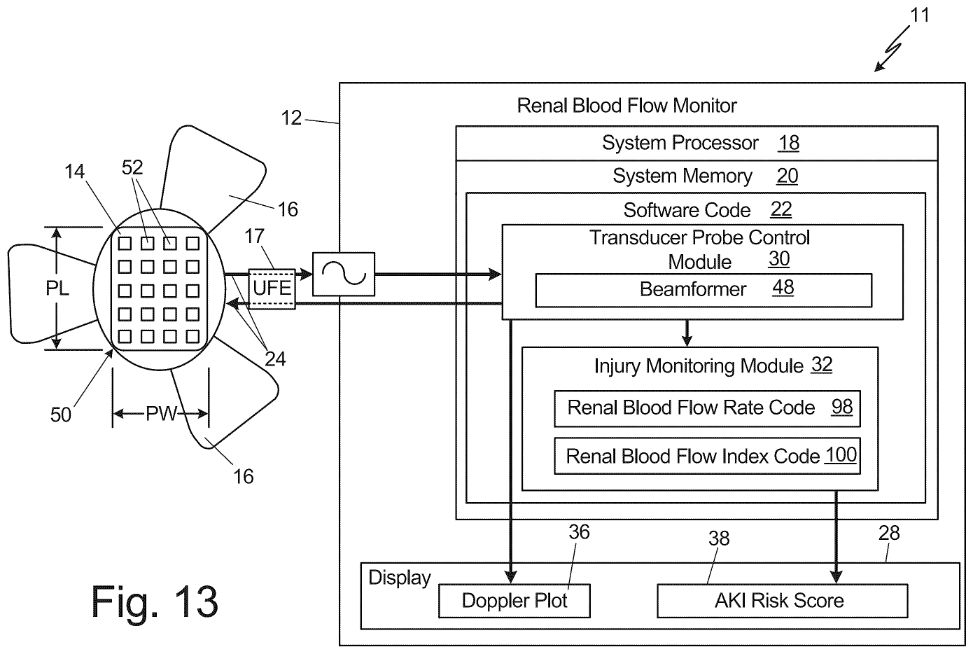

FIG. 13 is a schematic diagram of monitoring system 11 from FIGS. 1 and 2 with injury monitoring module 32 of software code 22 further including the following two software sub-modules: renal blood flow rate monitoring software code 98 and renal blood flow index monitoring software code 100. When system processor 18 executes injury monitoring module 32, injury monitoring module 32 can use any or both of renal blood flow rate monitoring software code 98 and renal blood flow index monitoring software code 100 as characteristics of the renal blood flow to determine, in real time, the AKI risk score of patient 10 from the Doppler flow signal DF of the renal blood flow of kidney 42L.

Renal blood flow rate monitoring software code 98 includes software code that first estimates, when executed by system processor 18, a baseline flow rate value for the renal blood flow of kidney 42L of patient 10 from the Doppler flow signal DF. System processor 18 can also execute renal blood flow rate monitoring software code 98 to generate a real-time renal flow rate value for the renal blood flow and continuously output the real-time renal flow rate value to display 28. System processor 18 can also execute renal blood flow rate monitoring software code 98 to track a running sum of the time the real-time renal flow rate value is below the baseline flow rate value. System processor 18 can also execute renal blood flow rate monitoring software code 98 to track how low or deep the real-time renal blood flow rate falls below the baseline flow rate value over time throughout a surgery, medical procedure, or medical observation of patient 10. Injury monitoring module 32 can utilize the running sum of the time the real-time renal flow rate value is below the baseline flow rate value as well as the low real-time renal blood flow rate values as variables in estimating the AKI risk score of patient 10.

Renal blood flow index monitoring software code 100 includes software code that first estimates, when executed by system processor 18, a real-time renal blood flow index from the Doppler flow signal DF of the renal blood flow of kidney 42L and continuously outputs the real-time renal blood flow index to display 28. The real-time renal blood flow index can be estimated without normalization from various Doppler flow characteristics such as the intensity-weighted total or mean flow velocity over time, or the peak flow velocity.

Normalized blood flow indices are useful. Renal blood flow index monitoring software code 100 can use the Renal Resistive Index (RRI) to calculate a normalized real-time renal blood flow index from the renal artery flow. The system processor 18 and renal blood flow index monitoring software code 100 can use Equation 1 to determine RRI from the Doppler flow signal DF of the renal blood flow of kidney 42L:

$$RRI = \frac{( peak systolic velocity - end diastolic velocity )}{peak systolic velocity} . \qquad Equation 1$$

Renal blood flow index monitoring software code 100 can also use the Venous Impedance Index (VII) to calculate a normalized real-time renal blood flow index from the Doppler flow signal DF of the renal blood flow in the renal vein of kidney 42L. System processor 18 and renal blood flow index monitoring software code 100 can use Equation 2 to determine VII from the Doppler flow signal DF of the renal blood flow of kidney 42L:

$$VII = \frac{( maximum flow velocity - minimum flow velocity )}{maximum flow velocity} . \qquad Equation 2$$

After calculating the real-time renal blood flow index for the renal blood flow of kidney 42L, system processor 18 can also execute renal blood flow index monitoring software code 100 to generate a baseline index value for the renal blood flow. For RRI, a value between 0.50-0.70 is considered a normal value. System processor 18 and renal blood flow index monitoring software code 100 can determine the baseline index value by monitoring the real-time renal blood flow index while patient 10 is under normal healthy conditions, or by choosing a baseline value established by past clinical studies, such as selecting a baseline RRI of 0.50-0.70. An RRI value above 0.70 can be indicative organ damage to kidney 42L.

System processor 18 can execute renal blood flow index monitoring software code 100 to track a running sum of the time the real-time renal blood flow index is above the baseline index value. System processor 18 can also execute renal blood flow index monitoring software code 100 to track how high the real-time renal blood flow index exceeds the baseline index value over time. Injury monitoring module 32 can utilize the running sum of the time the real-time renal blood flow index is above the baseline index value as wells as the high real-time renal blood flow index values as variables in estimating the AKI risk score of patient 10.

The renal blood flow monitor can use the continuous Doppler flow measured in the renal vein or hepatic vein or portal vein to measure the Venous Excess Ultrasound Score (VEXUS) from the Doppler flow waveform characteristics in these vessels. The blood flow monitor can continuously measure the VEXUS. The VEXUS is useful clinical information for determining venous congestion and elevated right atrium pressure.

### Discussion of Possible Embodiments

The following are non-exclusive descriptions of possible embodiments of the present invention.

A renal blood flow monitor includes an ultrasound transducer probe with a two-dimensional array of transducer elements. An adhesive patch is connected to the ultrasound transducer probe and is configured to attach the ultrasound transducer probe to a patient and maintain contact between the patient and the ultrasound transducer probe without an operator. A beamformer drives the two-dimensional array of transducer elements is configured to cause the two-dimensional array of transducer elements to emit multiple ultrasound beams from the two-dimensional array of transducer elements to track a Doppler flow signal of a renal blood flow of the patient relative to the array of transducer elements.

The renal blood flow monitor of the preceding paragraph can optionally include, additionally and/or alternatively, any one or more of the following features, configurations and/or additional components in the paragraphs below.

In an embodiment of the foregoing renal blood flow monitor, the renal blood flow monitor further comprises a system memory that stores monitoring software code; and a processor configured to execute the monitoring software code to: determine a characteristic associated with the renal blood flow of the patient; and monitor over time the characteristic associated with the renal blood flow of the patient.

In an embodiment of the foregoing renal blood flow monitor, the renal blood flow monitor further comprises a display in communication with the processor to receive and show a continuous reading of the Doppler flow signal from the ultrasound transducer probe and a representation of the characteristic associated with the renal blood flow of the patient.

In an embodiment of the foregoing renal blood flow monitor, the monitoring software code comprises: renal blood flow index monitoring software code, and wherein the processor is configured to execute the renal blood flow index monitoring software code to: estimate a renal blood flow index from the Doppler flow signal of the renal blood flow; and establish a baseline value for the renal blood flow index of the patient from the Doppler flow signal sensed by the ultrasound transducer probe.

In an embodiment of the foregoing method, the processor is further configured to execute the renal blood flow index monitoring software code to: output to the display a representation of the renal blood flow index of the patient.

In an embodiment of the foregoing method, the renal blood flow index comprises a Venous Impedance Index (VII), a Renal Resistive Index (RRI), and/or a Venous Excess Ultrasound (VExUS) score.

In an embodiment of the foregoing method, the monitoring software code comprises: renal blood flow rate monitoring software code, and the processor is configured to execute the renal blood flow rate monitoring software code to: estimate a renal blood flow rate from the Doppler flow signal of the renal blood flow; and output to the display a representation of the renal blood flow rate of the patient.

In an embodiment of the foregoing method, the monitoring software code comprises: acute kidney injury (AKI) monitoring software code, and the processor is configured to execute the AKI monitoring software code to: establish a baseline value for the renal blood flow of the patient from the Doppler flow signal of the renal blood flow sensed by the ultrasound transducer probe; continuously monitor the Doppler flow signal of the renal blood flow sensed by the ultrasound transducer probe throughout a duration of a surgery, medical procedure, or medical observation of the patient; estimate a real-time acute kidney injury risk score of the patient from the Doppler flow signal of the renal blood; and output to the display a representation of the real-time acute kidney injury risk score of the patient.

In an embodiment of the foregoing renal blood flow monitor, the renal blood flow monitor further comprises, an organ recognition algorithm configured to distinguish the renal blood flow signal from a non-renal blood flow signal based upon waveform characteristics of the renal blood flow signal.

In an embodiment of the foregoing renal blood flow monitor, the organ recognition algorithm comprises: a system memory that stores organ recognition software code; and a processor configured to execute the organ recognition software code to: perform waveform analysis of the Doppler flow signal of the patient sensed by the ultrasound transducer probe; extract waveform characteristics of the Doppler flow signal; compare the waveform characteristics of the Doppler flow signal to a reference table of renal blood flow waveform characteristics and non-renal blood flow waveform characteristics; and output a determination score that indicates whether the Doppler flow signal is from a renal blood flow or a non-renal blood flow.

In an embodiment of the foregoing renal blood flow monitor, the renal blood flow monitor further comprises a display in communication with the ultrasound transducer probe control and the organ recognition algorithm to receive and show a continuous reading of the Doppler flow signal from the ultrasound transducer probe and a representation of the determination score from the organ recognition algorithm.

In an embodiment of the foregoing renal blood flow monitor, the ultrasound transducer probe operates at a center frequency between 0.5 MHz and 4.0 MHz to penetrate more than 15 cm into the patient.

In an embodiment of the foregoing renal blood flow monitor, the array of transducer elements of the ultrasound transducer probe is sized, in at least one of the two dimensions, to cover one or more acoustic windows in the patient, wherein an acoustic window of the patient is defined as an area of the patient where transmission of ultrasonic waves is not substantially attenuated in comparison to immediate surroundings.

In an embodiment of the foregoing renal blood flow monitor, the array of transducer elements of the ultrasound transducer probe is sized in at least one of the two dimensions to extend over at least two intercostal spaces of the patient.

In an embodiment of the foregoing renal blood flow monitor, each transducer element in the array of transducer elements of the ultrasound transducer probe comprises an element width and length that are both larger than one wavelength in soft tissue of an ultrasonic wave emitted by the array of transducer elements.

In an embodiment of the foregoing renal blood flow monitor, the renal blood flow monitor further comprises a coupling layer comprising a couplant that enables ultrasonic energy transmission between a skin of the patient and the ultrasound transducer probe.

In an embodiment of the foregoing renal blood flow monitor, the renal blood flow monitor further comprises a system memory that stores the beamformer as flow signal tracking software code; and a processor configured to execute the flow signal tracking software code to: continuously monitor the Doppler flow signal of the renal blood flow sensed by the ultrasound transducer probe throughout a duration of a surgery, medical procedure, or medical observation of the patient.

A method is disclosed for monitoring renal blood flow of a patient. The method includes positioning an ultrasound transducer probe on an abdomen of the patient. The ultrasound transducer probe includes a two-dimensional array of transducer elements. The abdomen of the patient is scanned with the two-dimensional array of transducer elements and a beamformer drives the array of transducer elements to find and sense a Doppler flow signal of the renal blood flow of the patient. The ultrasound transducer probe is attached to the abdomen of the patient by an adhesive patch connected to the ultrasound transducer probe at a position on the abdomen of the patient where the Doppler flow signal of the renal blood flow of the patient was found. The beamformer and the array of transducer elements track-scan the Doppler flow signal of the renal blood flow of the patient to continuously sense the Doppler flow signal of the renal blood flow of the patient during a surgery, medical procedure, or medical observation without an ultrasound operator.

The method of the preceding paragraph can optionally include, additionally and/or alternatively, any one or more of the following features, configurations and/or additional components in the paragraphs below.

In an embodiment of the foregoing method, track-scanning the Doppler flow signal of the renal blood flow of the patient by the beamformer and the array of transducer elements comprises: emitting a set of sequential beams from the array of transducer elements to track a center of the renal blood flow relative to the array of transducer elements; focusing each beam from the set of beams in different locations; and adjusting the position of the set of beams onto the center of the renal blood flow by the beamformer to maintain the Doppler flow signal of the renal blood flow of the patient.

In an embodiment of the foregoing method, track-scanning the Doppler flow signal of the renal blood flow of the patient by the beamformer and the array of transducer elements comprises: measuring estimates of a location of the renal blood flow by the beamformer; inputting the estimates of the location of the renal blood flow into a predictive filter; and determining an expected trajectory of the location of the renal blood flow based on the estimates of the location of the renal blood flow and based on a breathing frequency of the patient.

In an embodiment of the foregoing method, measuring estimates of the location of the renal blood flow by the beamformer comprises: measuring, by the beamformer, differences in integrated power spectrum between individual beams of the set of sequential beams to estimate an azimuthal angle and an elevation angle of the location of the renal blood flow relative to the array of transducer elements; and estimating, by the beamformer, a distance of the blood flow from the array of transducer elements in a distance dimension by: gathering, by the array of transducer elements and the beamformer, a plurality of distance samples along a distance dimension; calculating, by the beamformer, integrated power spectrum for each distance sample of the plurality of distance samples; assigning, by the beamformer, a likelihood of containing the renal blood flow to each distance sample of the plurality of distance samples; and calculating, by the beamformer, an estimate of a center of the renal blood flow from the plurality of distance samples.

In an embodiment of the foregoing method, the method further comprises: making, by the beamformer, proportional the likelihood of containing the renal blood flow to the integrated power spectrum for each distance sample of the plurality of distance samples; and calculating, by the beamformer, the estimate of the center of the renal blood flow from the plurality of distance samples by selecting a distance sample of the plurality of distance samples with the largest integrated power spectrum.

In an embodiment of the foregoing method, the method further comprises: measuring the breathing frequency of the patient with a breathing monitor connected to the patient; and inputting the breathing frequency of the patient into the predictive filter from the breathing monitor.

In an embodiment of the foregoing method, the predictive filter comprises a Kalman Filter.

In an embodiment of the foregoing method, the method further comprises: continuously outputting a plot of the Doppler flow signal of the renal blood flow of the patient to a display in communication with the ultrasound transducer probe during the surgery, medical procedure, or medical observation without an ultrasound operator.

In an embodiment of the foregoing method, the method further comprises: communicating the Doppler flow signal sensed by the ultrasound transducer probe to a processor configured to execute monitoring software code stored on a system memory; determining, by the processor executing the monitoring software code, a characteristic associated with the renal blood flow of the patient from the Doppler flow signal of the renal blood flow sensed by the ultrasound transducer probe; and continuously monitoring, by the processor executing the monitoring software code, the Doppler flow signal of the renal blood flow and the characteristic associated with the renal blood flow of the patient during a surgery, medical procedure, or medical observation of the patient.

In an embodiment of the foregoing method, the method further comprises continuously outputting to a display in communication with the processor a plot of the Doppler flow signal of the renal blood flow of the patient and a representation of the characteristic associated with the renal blood flow of the patient during the surgery, medical procedure, or medical observation of the patient.

In an embodiment of the foregoing method, wherein the monitoring software code comprises: renal blood flow rate monitoring software code, and wherein the processor executes the renal blood flow rate monitoring software code to: estimate a renal blood flow rate from the Doppler flow signal of the renal blood flow; continuously monitor the renal blood flow rate during the surgery, medical procedure, or medical observation of the patient; and output to the display a representation of the renal blood flow rate of the patient over time.

In an embodiment of the foregoing method, the monitoring software code comprises: renal blood flow index monitoring software code, and wherein the processor executes the renal blood flow index monitoring software code to: estimate a renal blood flow index from the Doppler flow signal of the renal blood flow; establish a baseline value for the renal blood flow index of the patient from the Doppler flow signal sensed by the ultrasound transducer probe; continuously monitor the renal blood flow index during the surgery, medical procedure, or medical observation of the patient; and output to the display a representation of the renal blood flow index of the patient over time.

In an embodiment of the foregoing method, the monitoring software code comprises: Renal Resistive Index (RRI) monitoring software code, and wherein the processor executes the RRI monitoring software code to: estimate a RRI of the patient from the Doppler flow signal of the renal blood flow; establish a baseline value for RRI of the patient from the Doppler flow signal sensed by the ultrasound transducer probe; continuously monitor the RRI of the patient during the surgery, medical procedure, or medical observation of the patient; and output to the display a representation of the RRI of the patient over time.

In an embodiment of the foregoing method, the monitoring software code comprises: Venous Impedance Index (VII) monitoring software code, and wherein the processor executes the VII monitoring software code to: estimate a VII of the patient from the Doppler flow signal of the renal blood flow; establish a baseline value for VII of the patient from the Doppler flow signal sensed by the ultrasound transducer probe; continuously monitor the VII of the patient during the surgery, medical procedure, or medical observation of the patient; and output to the display a representation of the VII of the patient over time.

In an embodiment of the foregoing method, the monitoring software code comprises: Venous Excess Ultrasound (VExUS) monitoring software code, and wherein the processor executes the VExUS monitoring software code to: estimate a VExUS score of the patient from the Doppler flow signal of the renal blood flow; establish a baseline value for the VExUS score of the patient from the Doppler flow signal sensed by the ultrasound transducer probe; continuously monitor the VExUS score of the patient during the surgery, medical procedure, or medical observation of the patient; and output to the display a representation of the VExUS score of the patient over time.

In an embodiment of the foregoing method, the monitoring software code comprises: acute kidney injury (AKI) monitoring software code, and wherein the processor executes the AKI monitoring software code to: establish a baseline value for the renal blood flow of the patient from the Doppler flow signal of the renal blood flow sensed by the ultrasound transducer probe; continuously monitor the Doppler flow signal of the renal blood flow sensed by the ultrasound transducer probe throughout a duration of a surgery, medical procedure, or medical observation of the patient; estimate a real-time acute kidney injury risk score of the patient from the Doppler flow signal of the renal blood; and output to the display a representation of the real-time acute kidney injury risk score of the patient.

In an embodiment of the foregoing method, the method further comprises verifying, by the processor executing the monitoring software code, an identity of the Doppler flow signal of the renal blood flow of the patient by an organ recognition algorithm based upon waveform characteristics of the Doppler flow signal.

In an embodiment of the foregoing method, verifying, by the processor executing the monitoring software code, the identity of the Doppler flow signal of the renal blood flow of the patient by the organ recognition algorithm based upon waveform characteristics of the Doppler flow signal comprises: comparing, by the processor executing the monitoring software code, the waveform characteristics of the Doppler flow signal with a waveform reference table, wherein the waveform reference table is a table of renal blood flow waveform characteristics and non-renal blood flow waveform characteristics.

In an embodiment of the foregoing method, the waveform reference table is prepopulated with waveforms obtained from prior measurements from a population, and/or wherein the waveform reference table comprises information from the patient that is gathered in advance of the operation, medical procedure, or medical observation by scanning with ultrasound transducer probe each organ of the patient that will be monitored during the operation, medical procedure, or medical observation by the processor executing the monitoring software code.

In an embodiment of the foregoing method, the waveform characteristics of the Doppler flow signal comprise a Signal-to-Noise Ratio (SNR), an integrated power spectrum, a spectral envelope, a pulsatility, and/or a spectral bandwidth of the Doppler flow signal.

In an embodiment of the foregoing method, the method further comprises outputting to the display a quality grade/index that indicates a probability of the Doppler flow signal being from the renal blood flow of the patient or from a non-renal blood flow of the patient; and continuously communicating the quality grade/index as an input into the predictive filter during the operation, medical procedure, or medical observation.

In an embodiment of the foregoing method, the ultrasound transducer probe senses the Doppler flow signal of the renal blood flow of the patient from a renal artery of the patient, a renal vein of the patient, or from both the renal artery and the renal vein.

An organ blood flow monitor includes an ultrasound transducer probe with a two-dimensional array of transducer elements. An adhesive patch is connected to the ultrasound transducer probe and is configured to attach the ultrasound transducer probe to a patient. A beamformer drives the two-dimensional array of transducer elements and is configured to cause the two-dimensional array of transducer elements to emit multiple ultrasound beams from the two-dimensional array of transducer elements to track a targeted organ blood flow signal relative to the array of transducer elements.

The organ blood flow monitor of the preceding paragraph can optionally include, additionally and/or alternatively, any one or more of the following features, configurations and/or additional components in the paragraphs below.

In an embodiment of the foregoing organ blood flow monitor, the organ blood flow monitor further comprises: a system memory that stores targeted organ flow index monitoring software code; and a processor configured to execute the targeted organ flow index monitoring software code to: establish a baseline value for a targeted organ flow of the patient from the targeted organ blood flow signal sensed by the ultrasound transducer probe; continuously monitor the targeted organ blood flow signal sensed by the ultrasound transducer probe throughout a duration of a surgery on the patient; and estimate a real-time targeted organ blood flow index of the patient from the targeted organ blood flow signal.

In an embodiment of the foregoing organ blood flow monitor, the organ blood flow monitor further comprises a display in communication with the processor to receive and show a continuous reading of the targeted organ blood flow signal from the ultrasound transducer probe and a representation of the real-time targeted organ blood flow index of the patient.

In an embodiment of the foregoing organ blood flow monitor, the organ blood flow monitor further comprises: a system memory that stores organ blood flow rate monitoring software code; and a processor configured to execute the organ blood flow rate monitoring software code to: estimate a targeted organ blood flow rate from the targeted organ blood flow signal.

In an embodiment of the foregoing organ blood flow monitor, the organ blood flow monitor further comprises a display in communication with the processor to receive and show a continuous reading of the targeted organ blood flow signal from the ultrasound transducer probe and a representation of the targeted organ blood flow rate of the patient.

In an embodiment of the foregoing organ blood flow monitor, the organ blood flow monitor further comprises: a system memory that stores organ injury monitoring software code; and a processor configured to execute the organ injury monitoring software code to: establish a baseline value for an organ flow of the patient from the targeted organ blood flow signal sensed by the ultrasound transducer probe; continuously monitor the targeted organ blood flow signal sensed by the ultrasound transducer probe throughout a duration of a surgery on the patient; and estimate a real-time organ injury risk score of the patient from the targeted organ blood flow signal.

In an embodiment of the foregoing organ blood flow monitor, the organ blood flow monitor further comprises a display in communication with the processor to receive and show a continuous reading of the targeted organ blood flow signal from the ultrasound transducer probe and a representation of the real-time organ injury risk score of the patient.

In an embodiment of the foregoing organ blood flow monitor, the ultrasound transducer probe operates at a center frequency between 0.5 MHz and 4.0 MHz to penetrate more than 15 cm into the patient.

In an embodiment of the foregoing organ blood flow monitor, the array of transducer elements of the ultrasound transducer probe is sized, in at least one of the two dimensions, to cover one or more acoustic windows in the patient, wherein an acoustic window of the patient is defined as an area of the patient where transmission of ultrasonic waves is not substantially attenuated in comparison to immediate surroundings.

In an embodiment of the foregoing organ blood flow monitor, the array of transducer elements of the ultrasound transducer probe is sized in at least one of the two dimensions to extend over at least two intercostal spaces of the patient.

In an embodiment of the foregoing organ blood flow monitor, each transducer element in the array of transducer elements of the ultrasound transducer probe comprises an element width and length that are both larger than one wavelength in soft tissue of an ultrasonic wave emitted by the array of transducer elements.

In an embodiment of the foregoing organ blood flow monitor, the system memory and the processor further comprise organ recognition software code configured to distinguish the targeted organ blood flow signal from a non-targeted blood flow signal based upon waveform characteristics of the targeted organ blood flow signal.

In an embodiment of the foregoing organ blood flow monitor, the processor is configured to execute the organ recognition software code to: perform waveform analysis of a flow signal of the patient sensed by the ultrasound transducer probe; extract waveform characteristics of the flow signal; compare the waveform characteristics of the flow signal to a reference table of blood flow waveform characteristics of various organs; and output to the display a determination score that indicates whether the flow signal is from the targeted organ blood flow or a non-targeted blood flow.

In an embodiment of the foregoing organ blood flow monitor, the organ blood flow monitor further comprises a coupling layer comprising a couplant that enables ultrasonic energy transmission between a skin of the patient and the ultrasound transducer probe.

A method is disclosed for monitoring organ blood flow of a targeted organ of a patient during a surgery, medical procedure, or medical observation. The method includes positioning an ultrasound transducer probe on an abdomen of the patient, wherein the ultrasound transducer probe comprises a two-dimensional array of transducer elements. A targeted organ location of the patient is scanned with the two-dimensional array of transducer elements and a beamformer driving the array of transducer elements to find a Doppler flow signal of the organ blood flow of the patient. The ultrasound transducer probe is attached to the patient by an adhesive patch connected to the ultrasound transducer probe at the targeted organ location where the Doppler flow signal of the organ blood flow of the patient was found. The beamformer track-scans the Doppler flow signal of the organ blood flow of the patient to continuously sense the Doppler flow signal of the organ blood flow of the patient without repositioning the ultrasound transducer probe during the surgery, medical procedure, or medical observation.

The method of the preceding paragraph can optionally include, additionally and/or alternatively, any one or more of the following features, configurations and/or additional components in the paragraphs below.

In an embodiment of the foregoing method, track-scanning the Doppler flow signal of the organ blood flow of the patient by the beamformer and the array of transducer elements comprises: emitting a set of sequential beams from the array of transducer elements to track a center of the organ blood flow relative to the array of transducer elements; focusing each beam from the set of beams in different locations; and adjusting the position of the set of beams onto the center of the organ blood flow by the beamformer to maintain the Doppler flow signal of the organ blood flow of the patient.

In an embodiment of the foregoing method, the method further comprises continuously outputting a plot of the Doppler flow signal of the organ blood flow of the patient to a display in communication with the ultrasound transducer probe while the ultrasound transducer probe is attached to the patient by the adhesive patch.

In an embodiment of the foregoing method, the method further comprises: communicating the Doppler flow signal sensed by the ultrasound transducer probe to a processor configured to execute targeted organ blood flow index monitoring software code stored on a system memory; establishing, by the processor, a baseline value for the organ blood flow of the targeted organ from the Doppler flow signal of the targeted organ blood flow sensed by the ultrasound transducer probe; continuously monitoring, by the processor, the Doppler flow signal of the organ blood flow of the targeted organ sensed by the ultrasound transducer probe throughout a duration of the surgery, medical procedure, or medical observation; and estimating, by the processor, a real-time targeted organ blood flow index of the patient from the Doppler flow signal of the organ blood flow of the targeted organ.

In an embodiment of the foregoing method, the method further comprises: communicating the Doppler flow signal sensed by the ultrasound transducer probe to a processor configured to execute targeted organ injury monitoring software code stored on a system memory; establishing, by the processor, a baseline value for the organ blood flow of the targeted organ from the Doppler flow signal of the organ blood flow sensed by the ultrasound transducer probe; continuously monitoring, by the processor, the Doppler flow signal of the organ blood flow of the targeted organ sensed by the ultrasound transducer probe throughout a duration of the surgery, medical procedure, or medical observation; and estimating, by the processor, a real-time targeted organ injury risk score of the patient from the Doppler flow signal of the organ blood flow of the targeted organ of the patient.

In an embodiment of the foregoing method, the method further comprises continuously outputting to a display in communication with the processor a plot of the Doppler flow signal of the organ blood flow of the targeted organ of the patient and a representation of the real-time targeted organ blood flow index of the patient during the duration of the surgery, medical procedure, or medical observation.

In an embodiment of the foregoing method, the method further comprises continuously outputting to a display in communication with the processor a plot of the Doppler flow signal of the organ blood flow of the targeted organ of the patient and a representation of the real-time targeted organ injury risk score of the patient during the duration of the surgery, medical procedure, or medical observation.

In an embodiment of the foregoing method, the method further comprises verifying an identity of the Doppler flow signal of the organ blood flow of the patient by an organ recognition algorithm based upon waveform characteristics of the Doppler flow signal.

A renal blood flow monitor includes an ultrasound transducer probe and an adhesive patch connected to the ultrasound transducer probe for attaching the ultrasound transducer probe to a patient. The renal blood flow monitor further includes an organ recognition algorithm configured to distinguish a renal blood flow signal from a non-renal blood flow signal based upon waveform characteristics of the renal blood flow signal.

The renal blood flow monitor of the preceding paragraph can optionally include, additionally and/or alternatively, any one or more of the following features, configurations and/or additional components in the paragraphs below.

In an embodiment of the foregoing renal blood flow monitor, the ultrasound transducer probe comprises a two-dimensional array of transducer elements.

In an embodiment of the foregoing renal blood flow monitor, the renal blood flow monitor further comprises: a system memory that stores a beamformer with flow signal tracking software code; a processor configured to execute the beamformer and the flow signal tracking software code to: emit multiple beams from the array of transducer elements to track the Doppler flow signal of the renal blood flow relative to the array of transducer elements; and continuously monitor the Doppler flow signal of the renal blood flow sensed by the ultrasound transducer probe throughout a duration of a surgery, medical procedure, or medical observation of the patient; and a coupling layer on the ultrasound transducer probe comprising a couplant material for forming a contact between a skin and the ultrasound transducer probe.

In an embodiment of the foregoing renal blood flow monitor, the organ recognition algorithm comprises: a waveform reference table of renal blood flow waveform characteristics and non-renal blood flow waveform characteristics; and a waveform analyzer module that performs waveform analysis of a Doppler flow signal of the patient sensed by the ultrasound transducer probe, extracts waveform characteristics of the Doppler flow signal, and compares the waveform characteristics of the Doppler flow signal to the reference table, and outputs a determination score that indicates whether the Doppler flow signal is from the renal blood flow or the non-renal blood flow.

In an embodiment of the foregoing renal blood flow monitor, the renal blood flow monitor further comprises a display in communication with the ultrasound transducer probe, the beamformer, and the organ recognition algorithm to receive and show a continuous reading of the Doppler flow signal from the ultrasound transducer probe and the determination score from the organ recognition algorithm.

In an embodiment of the foregoing renal blood flow monitor, the renal blood flow monitor further comprises: a system memory that stores acute kidney injury (AKI) monitoring software code; and a processor configured to execute the AKI monitoring software code to: establish a baseline value for the renal blood flow of the patient from the Doppler flow signal of the renal blood flow sensed by the ultrasound transducer probe; continuously monitor the Doppler flow signal of the renal blood flow sensed by the ultrasound transducer probe throughout a duration of a surgery on the patient; estimate a real-time acute kidney injury risk score of the patient from the Doppler flow signal of the renal blood; and output a representation of the real-time acute kidney injury risk score of the patient to the display.

A renal blood flow monitor includes an ultrasound transducer probe with an array of transducer elements. An adhesive patch is connected to the ultrasound transducer probe and is configured to connect the ultrasound transducer probe to a patient. The renal blood flow monitor further includes a system memory that stores beamformer software code and a processor in communication with the ultrasound transducer probe and the system memory. The processor is configured to execute the beamformer software code to beam scan the patient with the array of transducer elements to find a Doppler flow signal of a renal blood flow of the patient.

The renal blood flow monitor of the preceding paragraph can optionally include, additionally and/or alternatively, any one or more of the following features, configurations and/or additional components in the paragraphs below.

In an embodiment of the foregoing renal blood flow monitor, the processor is configured to execute the beamformer software code to: track scan the renal blood flow of the patient by emitting multiple beams from the array of transducer elements to track a center of the renal blood flow relative to the array of transducer elements; and adjust the position of a beam of the beam scan onto the center of the renal blood flow to maintain the Doppler flow signal of the renal blood flow of the patient.

In an embodiment of the foregoing renal blood flow monitor, the renal blood flow monitor further comprises a display in communication with the ultrasound transducer probe and the processor to receive and show a continuous reading of the Doppler flow signal from the ultrasound transducer probe.

In an embodiment of the foregoing renal blood flow monitor, the ultrasound transducer probe operates at a center frequency between 0.5 MHz and 4.0 MHz to penetrate more than 15 cm into the patient.

In an embodiment of the foregoing renal blood flow monitor, the array of transducer elements of the ultrasound transducer probe is sized in length to cover one or more acoustic windows in the patient, wherein an acoustic window of the patient is defined as an area of the patient where transmission of ultrasonic waves is not substantially attenuated in comparison to immediate surroundings.

In an embodiment of the foregoing renal blood flow monitor, the array of transducer elements of the ultrasound transducer probe is sized in length to extend over at least two intercostal spaces of the patient.

In an embodiment of the foregoing renal blood flow monitor, each transducer element in the array of transducer elements of the ultrasound transducer probe comprises an element width and length that are both larger than one wavelength of an ultrasonic wave emitted by the array of transducer elements.

In an embodiment of the foregoing renal blood flow monitor, the array of transducer elements of the ultrasound transducer probe comprises a pitch defining an inter-element spacing between adjacent transducer elements, and wherein the pitch is larger than the one wavelength of the ultrasonic wave emitted by the array of transducer elements.

In an embodiment of the foregoing renal blood flow monitor, the system memory stores acute kidney injury (AKI) monitoring software code, and wherein the processor is configured to execute the AKI monitoring software code to: establish a baseline value for the renal blood flow of the patient from the Doppler flow signal of the renal blood flow sensed by the ultrasound transducer probe; continuously monitor the Doppler flow signal of the renal blood flow sensed by the ultrasound transducer probe throughout a duration of a surgery on the patient; estimate a real-time acute kidney injury risk score of the patient from the Doppler flow signal of the renal blood; and output a representation of the real-time acute kidney injury risk score to the display.

A blood flow monitor includes an ultrasound transducer probe with a two-dimensional array of transducer elements. An adhesive patch is connected to the ultrasound transducer probe and is configured to attach the ultrasound transducer probe to a patient. The blood flow monitor further includes both a system memory that stores beamformer software code and a processor in communication with the ultrasound transducer probe and the system memory. The processor is configured to execute the beamformer software code to steer a beam to scan the patient with the array of transducer elements to find a Doppler flow signal of a targeted blood flow of the patient.

The blood flow monitor of the preceding paragraph can optionally include, additionally and/or alternatively, any one or more of the following features, configurations and/or additional components in the paragraphs below.

In an embodiment of the foregoing blood flow monitor, the processor is configured to execute the beamformer software code to: track scan the targeted blood flow of the patient by emitting multiple beams from the array of transducer elements to track a center of the targeted blood flow relative to the array of transducer elements; and adjust the position of a beam of the beam scan onto the center of the targeted blood flow to maintain the Doppler flow signal of the targeted blood flow of the patient.

In an embodiment of the foregoing blood flow monitor, the blood flow monitor further comprises a display in communication with the ultrasound transducer probe and the processor to receive and show a continuous reading of the Doppler flow signal from the ultrasound transducer probe.

In an embodiment of the foregoing blood flow monitor, the ultrasound transducer probe operates at a center frequency between 0.5 MHz and 4.0 MHz to penetrate more than 15 cm into the patient.

In an embodiment of the foregoing blood flow monitor, the array of transducer elements of the ultrasound transducer probe is sized in length to cover one or more acoustic windows in the patient, wherein an acoustic window of the patient is defined as an area of the patient where transmission of ultrasonic waves is not substantially attenuated in comparison to immediate surroundings.

In an embodiment of the foregoing blood flow monitor, the array of transducer elements of the ultrasound transducer probe is sized in length to extend over at least two intercostal spaces of the patient.

In an embodiment of the foregoing blood flow monitor, each transducer element in the array of transducer elements of the ultrasound transducer probe comprises an element width and length that are both larger than one wavelength of an ultrasonic wave emitted by the array of transducer elements.

In an embodiment of the foregoing blood flow monitor, the array of transducer elements of the ultrasound transducer probe comprises a pitch defining an inter-element spacing between adjacent transducer elements, and wherein the pitch is larger than the one wavelength of the ultrasonic wave emitted by the array of transducer elements.

In an embodiment of the foregoing blood flow monitor, the system memory stores specific organ injury (SOI) monitoring software code, and wherein the processor is configured to execute the SOI monitoring software code to: establish a baseline value for the specific organ blood flow of the patient from the Doppler flow signal of the specific organ blood flow sensed by the ultrasound transducer probe; continuously monitor the Doppler flow signal of the specific organ blood flow sensed by the ultrasound transducer probe throughout a duration of a surgery on the patient; estimate a real-time specific organ injury risk score of the patient from the Doppler flow signal of the renal blood; and output a representation of the real-time specific organ injury risk score to the display.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A renal blood flow monitor, comprising:
an ultrasound transducer probe comprising a two-dimensional array of transducer elements;
an adhesive patch connected to the ultrasound transducer probe and configured to attach the ultrasound transducer probe to a patient and maintain contact between the patient and the ultrasound transducer probe without an operator; and
a beamformer driving the two-dimensional array of transducer elements, wherein the beamformer is configured to cause the two-dimensional array of transducer elements to emit multiple ultrasound beams from the two-dimensional array of transducer elements to track a Doppler flow signal of a renal blood flow of the patient relative to the array of transducer elements.

2. The renal blood flow monitor of claim 1, further comprising:
a system memory that stores monitoring software code; and
a processor configured to execute the monitoring software code to:
determine a characteristic associated with the renal blood flow of the patient; and
monitor over time the characteristic associated with the renal blood flow of the patient.

3. The renal blood flow monitor of claim 2, further comprising:
a display in communication with the processor to receive and show a continuous reading of the Doppler flow signal from the ultrasound transducer probe and a representation of the characteristic associated with the renal blood flow of the patient.

4. The renal blood flow monitor of claim 3, wherein the monitoring software code comprises:
renal blood flow index monitoring software code, and wherein the processor is configured to execute the renal blood flow index monitoring software code to:
estimate a renal blood flow index from the Doppler flow signal of the renal blood flow; and
establish a baseline value for the renal blood flow index of the patient from the Doppler flow signal sensed by the ultrasound transducer probe.

5. The renal blood flow monitor of claim 4, wherein the processor is further configured to execute the renal blood flow index monitoring software code to:
output to the display a representation of the renal blood flow index of the patient.

6. The renal blood flow monitor of claim 5, wherein the renal blood flow index comprises a Venous Impedance Index (VII), a Renal Resistive Index (RRI), and/or a Venous Excess Ultrasound (VExUS) score.

7. The renal blood flow monitor of claim 3, wherein the monitoring software code comprises:
renal blood flow rate monitoring software code, and the processor is configured to execute the renal blood flow rate monitoring software code to:
estimate a renal blood flow rate from the Doppler flow signal of the renal blood flow; and
output to the display a representation of the renal blood flow rate of the patient.

8. The renal blood flow monitor of claim 3, wherein the monitoring software code comprises:
acute kidney injury (AKI) monitoring software code, and the processor is configured to execute the AKI monitoring software code to:
establish a baseline value for the renal blood flow of the patient from the Doppler flow signal of the renal blood flow sensed by the ultrasound transducer probe;
continuously monitor the Doppler flow signal of the renal blood flow sensed by the ultrasound transducer probe throughout a duration of a surgery, medical procedure, or medical observation of the patient;
estimate a real-time acute kidney injury risk score of the patient from the Doppler flow signal of the renal blood; and
output to the display a representation of the real-time acute kidney injury risk score of the patient.

9. The renal blood flow monitor of claim 1, further comprising:
an organ recognition algorithm configured to distinguish the renal blood flow signal from a non-renal blood flow signal based upon waveform characteristics of the renal blood flow signal.

10. The renal blood flow monitor of claim 9, wherein the organ recognition algorithm comprises:
a system memory that stores organ recognition software code; and
a processor configured to execute the organ recognition software code to:
perform waveform analysis of the Doppler flow signal of the patient sensed by the ultrasound transducer probe;
extract waveform characteristics of the Doppler flow signal;
compare the waveform characteristics of the Doppler flow signal to a reference table of renal blood flow waveform characteristics and non-renal blood flow waveform characteristics; and
output a determination score that indicates whether the Doppler flow signal is from a renal blood flow or a non-renal blood flow.

11. The renal blood flow monitor of claim 10, further comprising:
a display in communication with the ultrasound transducer probe control and the organ recognition algorithm to receive and show a continuous reading of the Doppler flow signal from the ultrasound transducer probe and a representation of the determination score from the organ recognition algorithm.

12. The renal blood flow monitor of claim 1, wherein the array of transducer elements of the ultrasound transducer probe is sized, in at least one of the two dimensions, to cover one or more acoustic windows in the patient, wherein an acoustic window of the patient is defined as an area of the patient where transmission of ultrasonic waves is not substantially attenuated in comparison to immediate surroundings.

13. The renal blood flow monitor of claim 1, wherein the array of transducer elements of the ultrasound transducer probe is sized in at least one of the two dimensions to extend over at least two intercostal spaces of the patient.

14. The renal blood flow monitor of claim 12, wherein each transducer element in the array of transducer elements of the ultrasound transducer probe comprises an element width and length that are both larger than one wavelength in soft tissue of an ultrasonic wave emitted by the array of transducer elements.

15. The renal blood flow monitor of claim 1, further comprising:
a system memory that stores the beamformer as flow signal tracking software code; and
a processor configured to execute the flow signal tracking software code to:
continuously monitor the Doppler flow signal of the renal blood flow sensed by the ultrasound transducer probe throughout a duration of a surgery, medical procedure, or medical observation of the patient.

16. A method for monitoring renal blood flow of a patient, the method comprising:
positioning an ultrasound transducer probe on an abdomen of the patient, wherein the ultrasound transducer probe comprises a two-dimensional array of transducer elements;
scanning the abdomen of the patient with the two-dimensional array of transducer elements and a beamformer driving the array of transducer elements to find and sense a Doppler flow signal of the renal blood flow of the patient;
attaching the ultrasound transducer probe to the abdomen of the patient by an adhesive patch connected to the ultrasound transducer probe at a position on the abdomen of the patient where the Doppler flow signal of the renal blood flow of the patient was found; and
track-scanning the Doppler flow signal of the renal blood flow of the patient by the beamformer and the array of transducer elements to continuously sense the Doppler flow signal of the renal blood flow of the patient during a surgery, medical procedure, or medical observation without an ultrasound operator.

17. The method of claim 16, wherein track-scanning the Doppler flow signal of the renal blood flow of the patient by the beamformer and the array of transducer elements comprises:
emitting a set of sequential beams from the array of transducer elements to track a center of the renal blood flow relative to the array of transducer elements;
focusing each beam from the set of beams in different locations; and
adjusting the position of the set of beams onto the center of the renal blood flow by the beamformer to maintain the Doppler flow signal of the renal blood flow of the patient.

18. The method of claim 17, wherein track-scanning the Doppler flow signal of the renal blood flow of the patient by the beamformer and the array of transducer elements comprises:
measuring estimates of a location of the renal blood flow by the beamformer;
inputting the estimates of the location of the renal blood flow into a predictive filter; and
determining an expected trajectory of the location of the renal blood flow based on the estimates of the location of the renal blood flow and based on a breathing frequency of the patient.

19. The method of claim 18, wherein measuring estimates of the location of the renal blood flow by the beamformer comprises:
measuring, by the beamformer, differences in integrated power spectrum between individual beams of the set of sequential beams to estimate an azimuthal angle and an elevation angle of the location of the renal blood flow relative to the array of transducer elements; and
estimating, by the beamformer, a distance of the blood flow from the array of transducer elements in a distance dimension by:
gathering, by the array of transducer elements and the beamformer, a plurality of distance samples along a distance dimension;
calculating, by the beamformer, integrated power spectrum for each distance sample of the plurality of distance samples;
assigning, by the beamformer, a likelihood of containing the renal blood flow to each distance sample of the plurality of distance samples; and
calculating, by the beamformer, an estimate of a center of the renal blood flow from the plurality of distance samples.

20. The method of claim 19, wherein the method further comprises:
making, by the beamformer, proportional the likelihood of containing the renal blood flow to the integrated power spectrum for each distance sample of the plurality of distance samples; and
calculating, by the beamformer, the estimate of the center of the renal blood flow from the plurality of distance samples by selecting a distance sample of the plurality of distance samples with the largest integrated power spectrum.

21. The method of claim 18, further comprising:
measuring the breathing frequency of the patient with a breathing monitor connected to the patient; and
inputting the breathing frequency of the patient into the predictive filter from the breathing monitor.

22. The method of claim 16, further comprising:
continuously outputting a plot of the Doppler flow signal of the renal blood flow of the patient to a display in communication with the ultrasound transducer probe during the surgery, medical procedure, or medical observation without an ultrasound operator.

23. The method of claim 16, further comprising:
communicating the Doppler flow signal sensed by the ultrasound transducer probe to a processor configured to execute monitoring software code stored on a system memory;
determining, by the processor executing the monitoring software code, a characteristic associated with the renal blood flow of the patient from the Doppler flow signal of the renal blood flow sensed by the ultrasound transducer probe; and
continuously monitoring, by the processor executing the monitoring software code, the Doppler flow signal of the renal blood flow and the characteristic associated with the renal blood flow of the patient during a surgery, medical procedure, or medical observation of the patient.

24. The method of claim 23, further comprising:
continuously outputting to a display in communication with the processor a plot of the Doppler flow signal of the renal blood flow of the patient and a representation of the characteristic associated with the renal blood flow of the patient during the surgery, medical procedure, or medical observation of the patient.

25. The method of claim 24, wherein the monitoring software code comprises:
renal blood flow rate monitoring software code, and wherein the processor executes the renal blood flow rate monitoring software code to:
estimate a renal blood flow rate from the Doppler flow signal of the renal blood flow;
continuously monitor the renal blood flow rate during the surgery, medical procedure, or medical observation of the patient; and
output to the display a representation of the renal blood flow rate of the patient over time.

26. The method of claim 24, further comprising:
verifying, by the processor executing the monitoring software code, an identity of the Doppler flow signal of the renal blood flow of the patient by an organ recognition algorithm based upon waveform characteristics of the Doppler flow signal, by comparing, by the processor executing the monitoring software code, the waveform characteristics of the Doppler flow signal with a waveform reference table, wherein the waveform reference table is a table of renal blood flow waveform characteristics and non-renal blood flow waveform characteristics;
outputting to the display a quality grade/index that indicates a probability of the Doppler flow signal being from the renal blood flow of the patient or from a non-renal blood flow of the patient; and
continuously communicating the quality grade/index as an input into the predictive filter during the operation, medical procedure, or medical observation.
